(19) United States
(12) Patent Application Publication
(10) Pub. No.: US 2020/0095191 A1
(43) Pub. Date: Mar. 26, 2020

(54) PROCESS OF PREPARING IOSIMENOL

(71) Applicant: OTSUKA PHARMACEUTICAL CO., LTD., Tokyo (JP)

(72) Inventors: Jiri MALINAK, Jesenice (CZ); Ivan HLAVACEK, Praha (CZ); Petr ZAHRADNIK, Chomutov (CZ)

(21) Appl. No.: 16/609,412

(22) PCT Filed: Apr. 27, 2018
(86) PCT No.: PCT/JP2018/017192

§ 371 Date: Oct. 29, 2019

Related U.S. Application Data
(60) Provisional application No. 62/492,600, filed on May 1, 2017.

(51) Int. Cl. C07C 231/14; C07C 231/24

(52) U.S. Cl. CPC C07C 231/14; C07C 231/24

(57) ABSTRACT

The present invention relates to a 5-step process for preparing iosimenol starting from ammonium 3-amino-5-(aminocarbonyl)benzoate which is first converted to 3-amino-5-(aminocarbonyl)-2,4,6-triiodobenzoic acid using sodium iodine dichloride (NaI—Cl₂). The present invention further relates to processes for purifying iosimenol.

### TECHNICAL FIELD

The present invention generally relates to a process of preparing iosimenol and each step thereof.

### BACKGROUND ART

Iosimenol, N,N′-bis[3-carbamoyl-5-(2,3-dihydroxypropyl-carbamoyl)-2,4,6-triiodophenyl]-N,N′-b is (2,3-dihydroxypropyl)-malonamide, having the structure showed below, has been proposed as a useful nonionic X-ray contrast agent by Dr. Milos Sovak in 1995 (Patent Literature 1).

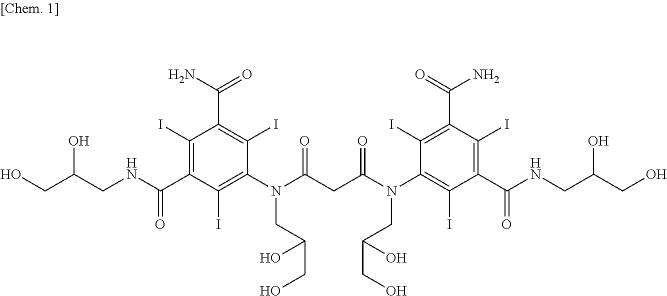 [SMILES: NC(=O)c1c(I)c(C(=O)NCC(O)CO)c(I)c(N(CC(O)CO)C(=O)CC(=O)N(CC(O)CO)c2c(I)c(C(N)=O)c(I)c(C(=O)NCC(O)CO)c2I)c1I]

In order to make iosimenol fit for commercial use as an X-ray contrast agent, it is necessary to manufacture iosimenol in a high yield and then purify the product effectively. Furthermore, an X-ray contrast agent is generally given to a human body in high dose, thus iosimenol as an X-ray contrast agent is specifically required to be in a high purity. However, it has been difficult to purify such a large amount of iosimenol effectively because iosimenol has chiral centers and pseudoasymmetric carbon atom in the bridge and chiral axes.

For example, Patent Literature 2 discloses some synthetic processes of iosimenol. In Patent Literature 2 (Example 9), iosimenol was prepared by reacting 5,5′-[(1,3-dioxo-1,3-propanediyl)diimino]bis[N-(2,3-dihydroxypropyl)-2,4,6-triiodo-1, 3-benzenedicarboxamide] (hereinafter, referred to as “C-VI”) with 3-chloro-propane-1,2-diol in water, but the yield was low and the purity of the product was low. Further, the crude iosimenol was purified through the following steps: deionization, ion-exchange resin adsorption, charcoaling finished by purification using LC reverse phase chromatography (Example 10 in Patent Literature 2). The average achieved HPLC purity was around 95% when starting from 85% deionized crude iosimenol.

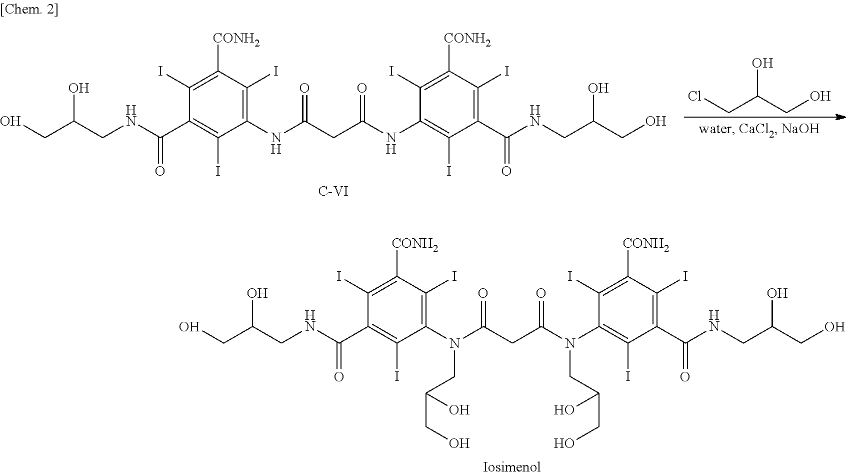 [SMILES: NC(=O)c1c(I)c(NC(=O)CC(=O)Nc2c(I)c(C(N)=O)c(I)c(C(=O)NCC(O)CO)c2I)c(I)c(C(=O)NCC(O)CO)c1I ; OCC(O)CCl ; "C-VI" ; NC(=O)c1c(I)c(C(=O)NCC(O)CO)c(I)c(N(CC(O)CO)C(=O)CC(=O)N(CC(O)CO)c2c(I)c(C(N)=O)c(I)c(C(=O)NCC(O)CO)c2I)c1I]

In addition, Patent Literature 1 also discloses a process of preparing iosimenol as shown below. In the process, C-VI was protected with isopropylidene beforehand, and then the protected C-VI (C-VI diacetonide) was reacted with 3-chloro-propane-1,2-diol in methanol. The present inventors actually reviewed the process, but both of the yield and the purity were low.

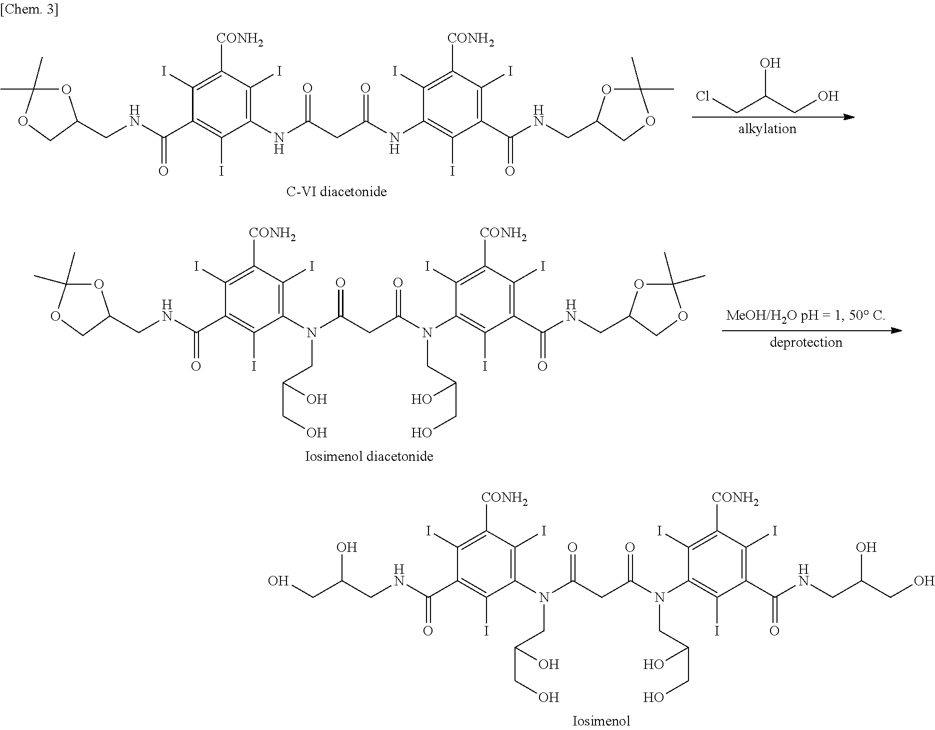

As mentioned above, it is important to manufacture iosimenol in a high purity for a commercial purpose, thus it is necessary to purify a crude product of iosimenol in some way. For manufacturing process, however, there are some real limitations to purify iosimenol using HPLC. It is evident, that the HPLC method represents a powerful and efficient method to reach purity requirements. On the other hand, the HPLC methods have many disadvantages such as low yield, extremely high amount of aqueous waste containing organic solvents (1,000 kg per kg of purified material), and enormously high investment costs. Combination of all these disadvantages constitutes outstandingly high production costs.

### CITATION LIST

### Patent Literature

[PL 1] U.S. Pat. No. 5,698,739 B

[PL 2] WO 2009/091758

### SUMMARY OF INVENTION

### Technical Problem

The main purpose of the present invention is to provide an effective preparation and/or purification of iosimenol in a high yield and in a high purity.

### Solution to Problem

The present inventors have intensively studied to carry out the above purpose and then found specific conditions of the process of preparing iosimenol from C-II in a high yield and in a high purity, as well as specific conditions of the purifications. Based upon the new findings, the present invention has been completed.

The present invention provides processes to prepare iosimenol and each step thereof as shown in the following Term 1 to Term 10.

Term 1. A process of preparing iosimenol shown in the following scheme:

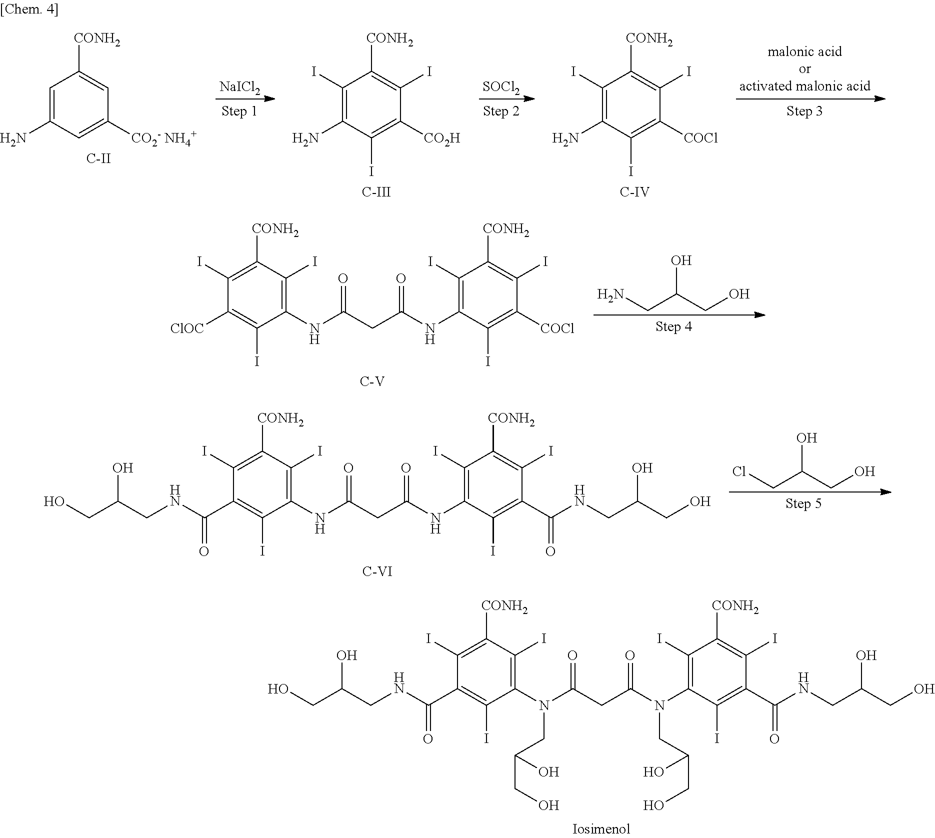

Term 2. The process of Term 1, wherein the crude product C-III in Step 1 is purified by crystallization in a solvent comprising methanol and/or a mixture of methanol and water (methanol: 1-99 wt %) at 20 to 100° C.

Term 3. The process of Term 1 or 2, wherein C-III in Step 2 is chlorinated with thionyl chloride in a solvent comprising ethyl acetate and/or toluene at reflux in the presence or without of catalytic amount of N,N-dimethylformamide.

Term 4. The process of any one of Terms 1 to 3, wherein crude C-IV in Step 2 is purified using an anion exchange resin to remove organic impurities, where the resin is a polystyrene-based resin, a polyacrylate-based resin, preferably a benzene ethylene-divinylbenzene copolymer-based resin (a styrene-divinylbenzene copolymer-based resin).

Term 5. The process of any one of Terms 1 to 4, wherein C-IV in Step 3 is coupled with malonic acid in the presence of phosphorus trichloride.

Term 6. The process of any one of Terms 1 to 4, wherein C-IV in Step 3 is coupled with an activated malonic acid.

Term 7. The process of Term 6, wherein as the activated malonic acid can be used its reactive ester or mixed anhydride which is in-situ prepared preferably by the addition of dicyclohexylcarbodiimide/N-hydroxybenztriazole and/or dicyclohexylcarbodiimide/hydroxysuccinimide and/or 1-ethyl-3-(3-dimethylaminopropyl)carbodiimide.HCl/N-hydroxybenztriazole and/or 1-propanephosphonic acid cyclic anhydride.

Term 8. The process of any one of Term 5 to 7, wherein the reaction is done in freshly distilled tetrahydrofuran or methyltetrahydrofuran.

Term 9. The process of any one of Terms 5 to 8, wherein the crude product C-V is purified by stirring in a solvent comprising tetrahydrofuran, methyltetrahydrofuran, diethyl ether, dioxane, or a mixture thereof.

Term 10. The process of any one of Terms 5 to 8, wherein the C-V is not dried and is used directly in the next step (Step 4).

Term 11. The process of any one of Terms 1 to 10, wherein C-V in Step 4 reacts with 3-amino-propane-1,2-diol in an organic solvent in the presence of a base at 2-25° C.

Term 12. The process of Term 11, wherein the organic solvent is N,N-dimethylformamide, in the presence of triethylamine.

Term 13. The process of Term 11 or 12, wherein the C-VI reaction mixture is stirred with an anion exchange resin suspended in aqueous methanol to separate organic impurities.

Term 14. The process of Term 13, wherein the anion exchange resin is a polystyrene-based resin, a polyacrylate-based resin, preferably a benzene ethylene-divinylbenzene copolymer-based resin (a styrene-divinylbenzene copolymer based resin).

Term 15. The process of any one of Terms 11 to 14, wherein C—VI is separated and purified directly by precipitation from the reaction mixture by the addition of an organic solvent selected from methanol, ethanol, n-propanol, 2-propanol, or a combination thereof under pH 5-7.

Term 16. The process of any one of Terms 11 to 15, wherein the precipitated C-VI is crystallized from a solvent mixture consisting water, acetone and acetic acid.

Term 17. The process of any one of Terms 1 to 16, wherein C-VI reacts in Step 5 with an alkylating agent introducing 2,3-dihydroxypropyl group in the presence of an inorganic base in an organic solvent selected from N,N-dimethylformamide, N,N-dimethylacetamide, dimethyl sulfoxide, N-methyl-2-pyrrolidone, ethylene glycol, propylene glycol, glycerine, methanol, or a combination thereof in the presence of 2-methoxyethanol (0-99%).

Term 18. The process of Term 17, wherein the alkylating agent introducing 2,3-dihydroxypropyl group is selected from the group consisting of 3-halo-propane-1,2-diol and glycidol.

Term 19. The process of Term 17 or 18, wherein the alkylating agent introducing 2,3-dihydroxypropyl group is 3-halo-propane-1,2-diol.

Term 20. The process of any one of Terms 17 to 19, wherein the alkylating agent introducing 2,3-dihydroxypropyl group is added into the stirred reaction mixture in one or more portion during reaction time.

Term 21. The process of any one of Terms 17 to 20, wherein the reaction temperature is 10-60° C.

Term 22. The process of any one of Terms 17 to 21, wherein the inorganic base is selected from the group consisting of an alkali metal hydroxide and an alkaline earth metal hydroxide.

Term 23. The process of any one of Terms 17 to 22, wherein the inorganic base is lithium hydroxide, calcium hydroxide, sodium hydroxide, potassium hydroxide, or a mixture thereof.

Term 24. The process of any one of Terms 17 to 23, wherein the reaction to prepare iosimenol is done in the presence of a metal halide besides an inorganic base.

Term 25. The process of Term 24, wherein the metal halide is selected from the group consisting of CaCl₂, ZnCl₂, MgCl₂, CaBr₂, ZnBr₂ and MgBr₂.

Term 26. A process of preparing C-III shown in the following scheme:

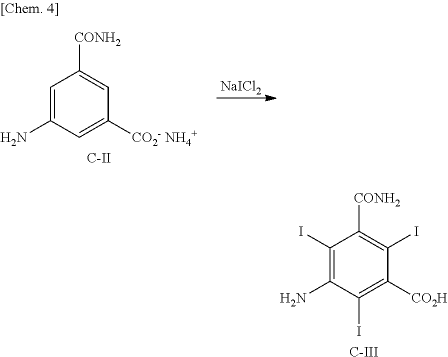

wherein the crude product C-III is purified by crystallization in a solvent comprising methanol or a mixture of methanol and water (methanol: 1-99 wt %) at 20 to 100° C.

Term 27. A crystal of C-III methanol solvate of the following formula:

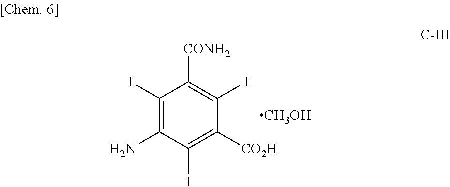

which is characterized by a powder x-ray diffraction pattern having four or more 2θ±0.2 peaks and selected from about 12.2°, 12.8°, 15.0°, 21.1°, 21.4°, 22.7°, 24.6°, 25.3°, 27.2°, 31.0°, 31.2°, 33.4°, and 33.9°, wherein measurement of said crystal is at a temperature of about 293 K.

Term 28. A crystal of C-III methanol solvate of the following formula:

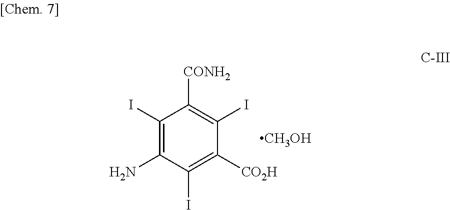

which is characterized by unit cell parameters at T=293K substantially equal to the following: a=17.000 (1) Å, b=13.896 (1) Å, c=12.597 (1) Å, unit-cell volume V=2975.9 Å³ and an orthorhombic space group Pbca.

Term 29. A process of preparing C-IV shown in the following scheme:

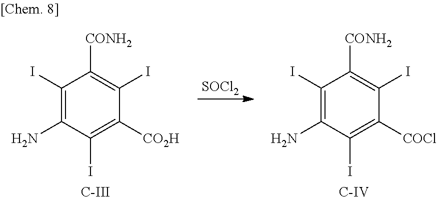

wherein C—III is chlorinated with thionyl chloride in a solvent comprising ethyl acetate and/or toluene at reflux in the presence or without of catalytic amount of N,N-dimethylformamide.

Term 30. The process of Term 29, wherein the crude C-IV is purified using an anion exchange resin to remove organic impurities, where the resin is a polystyrene-based resin, a polyacrylate-based resin, preferably a benzene ethylene-divinylbenzene copolymer-based resin (a styrene-divinylbenzene copolymer based resin).

Term 31. A process of preparing C-V shown in the following scheme:

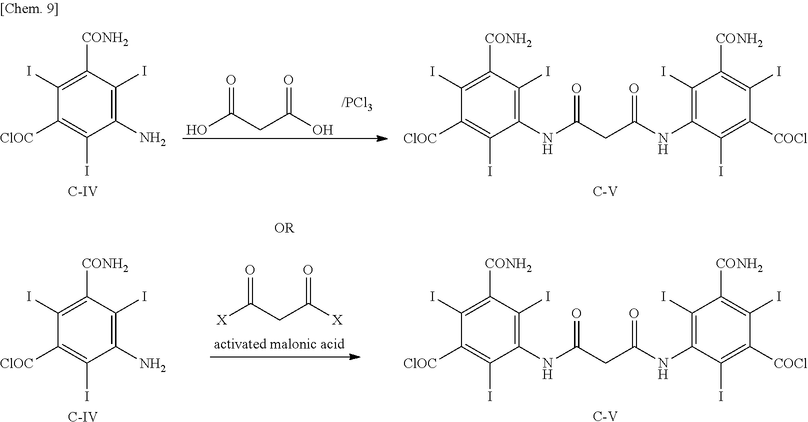

wherein C—IV is coupled with malonic acid in the presence of phosphorus trichloride, or with an activated malonic acid.

Term 32. The process of Term 31, wherein C—IV is coupled with an activated malonic acid.

Term 33. The process of Term 32, wherein as the activated malonic acid can be used its reactive ester or mixed anhydride which is in-situ prepared preferably by addition of dicyclohexylcarbodiimide/N-hydroxybenztriazole and/or dicyclohexylcarbodiimide/hydroxysuccinimide and/or 1-ethyl-3-(3-dimethylaminopropyl)carbodiimide.HCl/N-hydroxybenztriazole and/or 1-propanephosphonic acid cyclic anhydride.

Term 34. The process of any one of Terms 31 to 33, wherein the reaction is done in freshly distilled tetrahydrofuran or methyltetrahydrofuran.

Term 35. The process of any one of Terms 31 to 34, wherein the crude product C-V is purified by stirring in a solvent comprising tetrahydrofuran, methyltetrahydrofuran, diethyl ether, dioxane or a mixture thereof.

Term 36. The process of any one of Terms 31 to 35, wherein the C-V is not dried and is used directly in the next reaction step.

Term 37. A process of preparing C-VI shown in the following scheme:

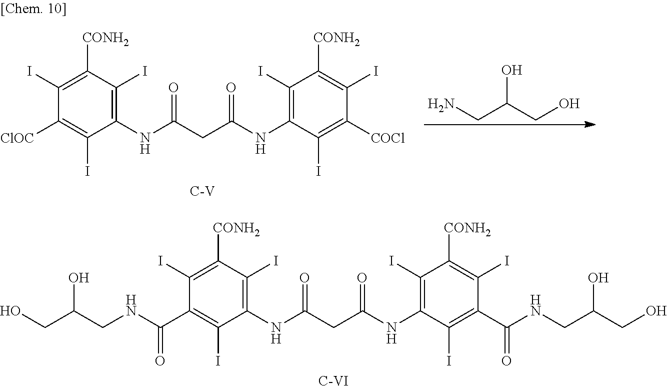

wherein C-V reacts with 3-amino-propane-1,2-diol in organic solvent in the presence of a base at 2-25° C.

Term 38. The process of Term 37, wherein the organic solvent is N,N-dimethylformamide, in the presence of trimethylamine.

Term 39. The process of Term 37 or 38, wherein C-VI reaction mixture is stirred with an anion exchange resin suspended in aqueous methanol to separate organic impurities.

Term 40. The process of Term 39, wherein the anion exchange resin is a polystyrene-based resin, a polyacrylate-based resin, preferably a benzene ethylene-divinylbenzene copolymer-based resin (a styrene-divinylbenzene copolymer based resin).

Term 41. The process of any one of Terms 37 to 40, wherein C—VI is separated and purified directly by precipitation from the reaction mixture by addition of an organic solvent selected from methanol, ethanol, n-propanol, 2-propanol or a combination thereof under pH 5-7.

Term 42. The process of any one of Terms 37 to 41, wherein precipitated C-VI is crystallized from a solvent mixture consisting water, acetone and acetic acid.

Term 43. A process of preparing iosimenol shown in the following scheme:

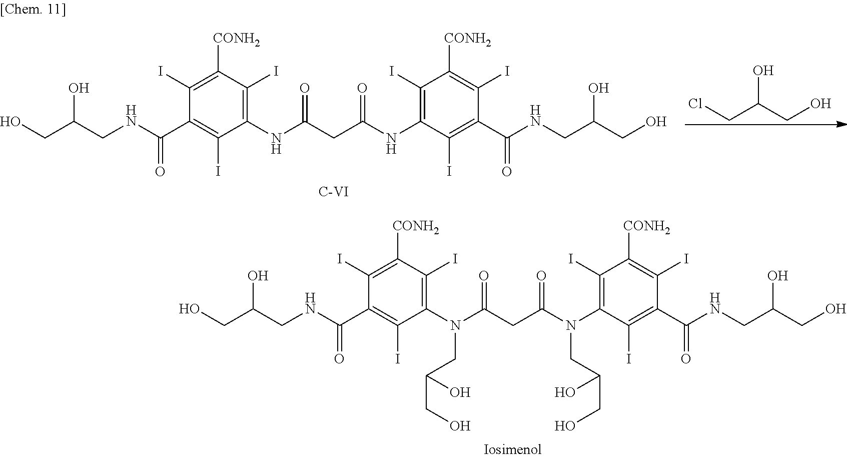 [SMILES: NC(=O)c1c(I)c(NC(=O)CC(=O)Nc2c(I)c(C(N)=O)c(I)c(C(=O)NCC(O)CO)c2I)c(I)c(C(=O)NCC(O)CO)c1I ; OCC(O)CCl ; "C-VI" ; NC(=O)c1c(I)c(C(=O)NCC(O)CO)c(I)c(N(CC(O)CO)C(=O)CC(=O)N(CC(O)CO)c2c(I)c(C(N)=O)c(I)c(C(=O)NCC(O)CO)c2I)c1I]

wherein C-VI reacts with an alkylating agent introducing 2,3-dihydroxypropyl group in the presence of an inorganic base in an organic solvent selected from N,N-dimethylformamide, N,N-dimethylacetamide, dimethyl sulfoxide, N-methyl-2-pyrrolidone, ethylene glycol, propylene glycol, glycerine, methanol or a combination thereof in the presence of 2-methoxyethanol (0-99%).

Term 44. The process of Term 43, wherein the alkylating agent introducing 2,3-dihydroxypropyl group is selected from the group consisting of 3-halo-propane-1,2-diol and glycidol.

Term 45. The process of Term 43 or 44, wherein the alkylating agent introducing 2,3-dihydroxypropyl group is 3-halo-propane-1,2-diol.

Term 46. The process of any one of Terms 43 to 45, wherein the alkylating agent introducing 2,3-dihydroxypropyl group is added into the stirred reaction mixture in one or more portion during the reaction time.

Term 47. The process of any one of Terms 43 to 46, wherein the reaction temperature is 10-60° C.

Term 48. The process of any one of Terms 43 to 47, wherein the inorganic base is selected from the group consisting of an alkali metal hydroxide and an alkaline earth metal hydroxide.

Term 49. The process of any one of Terms 43 to 48, wherein the inorganic base is lithium hydroxide, calcium hydroxide, sodium hydroxide, potassium hydroxide or a mixture thereof.

Term 50. The process of any one of Terms 43 to 49, wherein the reaction to prepare iosimenol is done in the presence of a metal halide besides an inorganic base.

Term 51. The process of Term 50, wherein the metal halide is selected from the group consisting of CaCl₂, ZnCl₂, MgCl₂, CaBr₂, ZnBr₂ and MgBr₂.

Term 52. A process of preparing iosimenol shown in the following scheme:

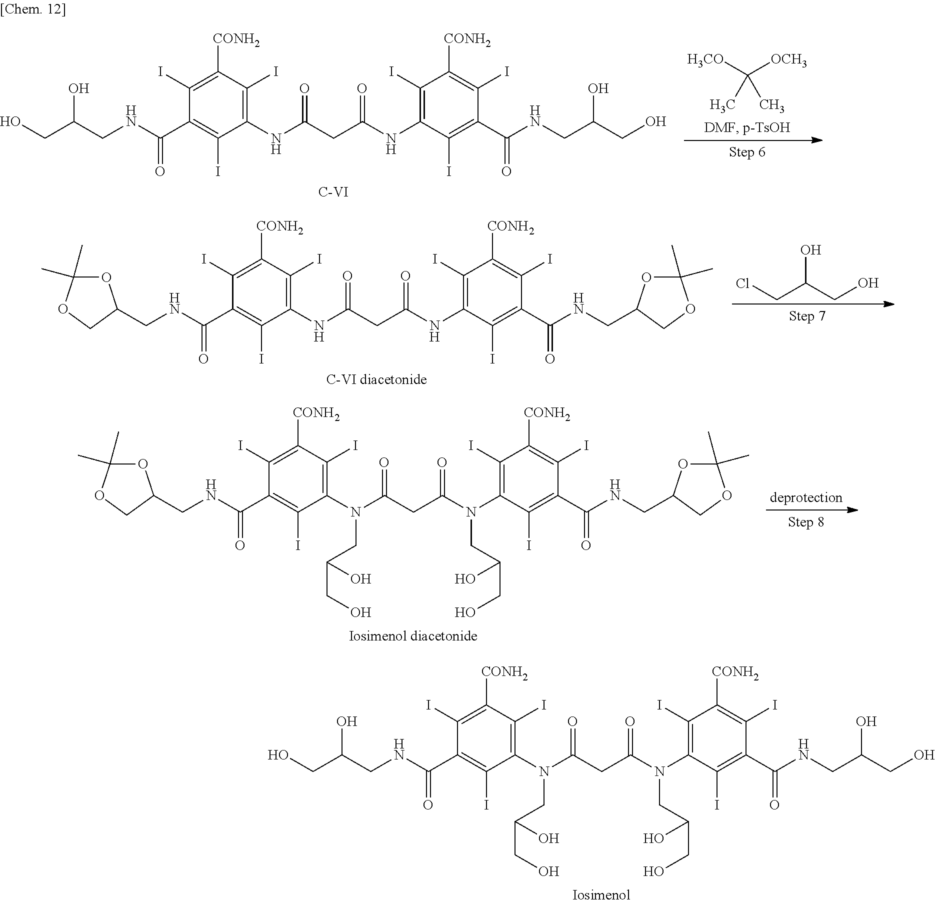

Term 53. The process of Term 52, wherein C—VI is protected with 2,2-dimethoxypropane in N,N-dimethylformamide, in the presence of an acidic catalyst.

Term 54. The process of Term 52 or 53, wherein C-VI diacetonide is crystallized from the reaction mixture by adding water.

Term 55. The process of any one of Terms 52 to 54, wherein a crystal of C-VI diacetonide is obtained in high purity (+98%) and high yield (+92%).

Term 56. The process of any one of Terms 52 to 55, wherein C-VI diacetonide reacts with an alkylating agent introducing 2,3-dihydroxypropyl group in the presence of an inorganic base in an organic solvent selected from N,N-dimethylformamide, N,N-dimethylacetamide, dimethyl sulfoxide, N-methyl-2-pyrrolidone, ethylene glycol, propylene glycol, glycerine, methanol, or a combination thereof, and/or their mixtures with methoxyethanol (0-99%).

Term 57. The process of any one of Terms 52 to 56, wherein the alkylating agent introducing 2,3-dihydroxypropyl group is selected from the group consisting of 3-halo-propane-1,2-diol and glycidol.

Term 58. The process of any one of Terms 52 to 57, wherein the alkylating agent introducing 2,3-dihydroxypropyl group is 3-halo-propane-1,2-diol.

Term 59. The process of any one of Terms 52 to 58, wherein the alkylating agent introducing 2,3-dihydroxypropyl group is added into the stirred reaction mixture in one or more portion during the reaction time.

Term 60. The process of any one of Terms 52 to 59, wherein the reaction temperature is 10-60° C.

Term 61. The process of any one of Terms 52 to 60, wherein the inorganic base is selected from the group consisting of an alkali metal hydroxide and an alkaline earth metal hydroxide.

Term 62. The process of any one of Terms 52 to 61, wherein the inorganic base is lithium hydroxide, calcium hydroxide, sodium hydroxide, potassium hydroxide, or a mixture thereof.

Term 63. The process of any one of Terms 52 to 62, wherein the reaction to prepare iosimenol diacetonide is done in the presence of a metal halide besides an inorganic base.

Term 64. The process of Term 63, wherein the metal halide is selected from the group consisting of CaCl₂, ZnCl₂, MgCl₂, CaBr₂, ZnBr₂ and MgBr₂.

Term 65. The process of any one of Terms 52 to 64, wherein iosimenol diacetonide is obtained by crystallization from an organic solvent selected from methanol, ethanol, n-propanol, 2-propanol, or a combination thereof.

Term 66. The process of any one of Terms 52 to 65, wherein iosimenol is obtained by deprotection of iosimenol diacetonide in an aqueous or methanolic, or ethanolic solution, or a methanol-water or ethanol-water in the presence of a strong acid.

Term 67. A process of purifying iosimenol, wherein a crude iosimenol is purified by crystallization in (i) binary or tertiary solvent-mixture selected from 2-methoxyethanol, 1-methoxy-2-propanol, and alcohols which a selected from methanol, ethanol, 2-propanol, n-butanol and/or 2-butanol (ii) diethylene glycol and/or triethylene glycol, or (iii) 2-ethoxyethanol and/or 1-methoxy-2-propanol, in the presence of water.

Term 68. A process of purifying iosimenol which is done from a saturated or supersaturated solution of said compound comprising:

Step 1: suspending the deionized iosimenol in a solvent mixture comprising one or more organic solvent and water,

Step 2: subjecting the mixture to heat and/or ultrasonic to make the mixture completely dissolved,

Step 3: continuing to subject the solution to the same or different heat and/or ultrasonic to deposit a crystal,

Step 4: continuous addition of a solvent or a solvent mixture, or adding of a solvent or a solvent mixture in individual portions during the crystallization process (we increased the yield from 40-50% to 70-85%),

Step 5: collecting the resulting crystal on a filter.

Term 69. The process of Term 68, wherein the heating in Step 2 and/or Step 3 and/or Step 4 is done with microwave.

Term 70. The process of any one of Term 68 or 69, wherein the organic solvent in Step 1 and Step 4 comprises one or more C₁-C₆ linear or branched alkanols or alkoxyalkanols, C₂-C₈ aliphatic ethers, C₄-C₆ cyclic ethers, and/or glycols.

Term 71. The process of any one of Terms 68 to 70, wherein the organic solvent in Step 1 and Step 4 is selected from the group consisting of methanol, ethanol, n-propanol, 2-propanol, n-butanol, i-butanol, sec-butanol, tert-butanol, pentanols including isoamylalcohols, hexanols, 2-methoxyethanol, 2-ethoxyethanol, 1-methoxy-2-propanol, 2-isopropoxyethanol, ethylene glycol, diethylene glycol and triethylene glycol.

Term 72. The process of any one of Terms 68 to 71, wherein the solvent mixture in Step 1 and Step 4 contains up to 20% water.

Term 73. The process of any one of Terms 68 to 72, wherein the crystallization process in Step 3 may be initiated by adding a seed of iosimenol crystal while or after the temperature is raised.

Term 74. The process of any one of Terms 68 to 73, wherein trometamol is used to buffer pH during the crystallization process.

Term 75. The process of any one of Terms 68 to 74, wherein Steps 2, 3 and 4 are done at 70° C.-140° C. and at the pressure of 0-10 bars.

Term 76. The process of any one of Terms 68 to 75, wherein the concentration of iosimenol as the starting material in Step 1 and Step 4 is 10 w/v %-60 w/v %.

### Effect of Invention

The present invention provides effective processes of preparing iosimenol and each intermediate thereof of the present invention in a high yield. And, the present invention also provides effective purifications of iosimenol and each intermediate thereof in a high purity.

### BRIEF DESCRIPTION OF DRAWINGS

FIG. 1 shows an overview graph of thermogravimetric analysis of C-III methanol solvate in Example 3.

FIG. 2 shows X-ray powder diffraction pattern of C-III methanol solvate in Example 3 FIG. 3 shows mass spectrum of C-III methanol solvate in Example 3.

FIG. 4 shows an infrared spectrum of C-III methanol solvate in Example 3.

### DESCRIPTION OF EMBODIMENTS

Step 1

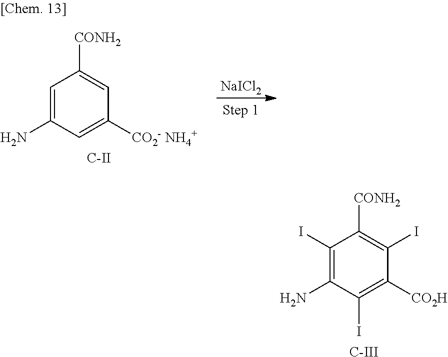

The current purification of crude intermediate C-III is not sufficiently effective for removal of impurities, which are responsible for formation of high amount of pentaiodo impurities in last step of synthesis of iosimenol; therefore, we developed crystallization of C-III in methanol via formation of C-III methanol solvate.

The above figure shows the synthetic pathway to intermediate C-III. The aqueous solution of ammonium salt of 5-amino-isophtalic acid, mono-primary amide is iodinated in water by the solution of sodium iodine dichloride at 75-80° C. Collected product is washed and purified through conversion into C-III sodium salt followed by its precipitation into C-III. The obtained C-III is dried.

C-III is purified by additional purification in methanol. The goal is obtaining C-III with HPLC purity higher than 99.0%. High purity of C-III is important for minimizing impurities in all synthetic steps leading to iosimenol API, mainly reduction of lower iodinated impurities (mono and di) and chloro impurities. The inventors have found that the purification in methanol is efficient. During the analysis of purified and dried C-III, we have found that dried C-III always contains 1-2% of residual solvents. We assumed, due to usage of large excess of methanol during purification, that methanol can be present in C-III molecule as a part of solvate with C-III. This assumption was confirmed as correct as shown in the working example shown below.

The described recrystallization of C-III from methanol yields C-III methanol solvate. This is a brand new molecule that has not yet been reported or published anywhere in scientific journals or patent literature, to the best of our knowledge (Reaxys and SciFinder databases), and we would be the first to patent it. The molar ratio C-III to methanol is approximately 1:0.94, as is apparent from NMR analysis in the working examples below. Producing C-III methanol solvate yields significantly purer C-III, thus minimizing impurities posing separation problems in subsequent steps in the preparation of Iosimenol. C-III methanol solvate is more crystalline than crude C-III as was confirmed by XRD in the working examples below. Exact structure and additional properties were further backed up with numerous analytical methods (melting point, density, TGA, MS, FTIR). See in the working examples below.

Step 2

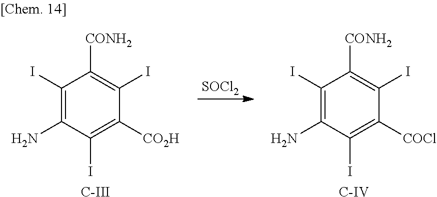

Impurities from step C-IV cause not only lower yields and higher amounts of impurities that have a negative impact on the impurity profile of intermediates in the next step, but also has negative influence on filterability of intermediates in next step. Generally, the best way how to prepare the intermediate with high purity and yield is usage of pure starting material. Preparation of 3-amino-5-(aminocarbonyl)-2,4,6-triiodobenzoyl chloride (C-IV) can be prepared by treatment of 3-amino-5-(aminocarbonyl)-2,4,6-triiodobenzoic acid C-III with thionyl chloride (U.S. Pat. No. 5,698,739, Carboxamide non-ionic contrast media) or in (U.S. Pat. No. 8,680,334, Process for the preparation of iosimenol). Both methods give low purity of intermediates C-IV (appx. 92%) both methods are described below. We developed preparation of intermediate C-IV with higher chromatographic purity (96-99% area). Unlike processes above (two patents), our strategy is based on achievement of significantly higher purity of C-IV and thus reduce transfer of impurities to following steps. Generally, higher purity of starting materials has also positive effect on yields in subsequent steps of iosimenol preparation.

Step 3

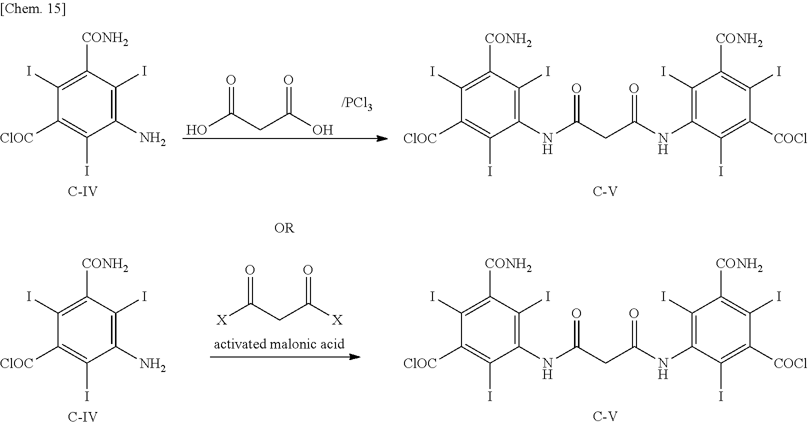

3,3′-[(1,3-dioxo-1,3-propanediyl)diimino]bis[5-(aminocarbonyl)-2,4,6-triiodobenzoyl chloride] (C-V) can be prepared by the treatment of 3-amino-5-(aminocarbonyl)-2,4,6-triiodobenzoyl chloride (C-IV) with malonyl dichloride as described in U.S. Pat. No. 5,698,739 (Carboxamide non-ionic contrast media) or in U.S. Pat. No. 8,680,334 (Process for the preparation of iosimenol). Neither of these patented syntheses provides high purity intermediate C-V. Also, environmental burden of chemical used in both patents is enormous. We developed two new processes for intermediate C-V:
1) process based on usage of phosphorus trichloride, when intensive washing of crude product is performed by tetrahydrofuran (THF).
2) process based on usage of activated malonic acid and its use ensures high purity and yields. Also, environmental burden is significantly reduced. The activation agent are dicyclohexylcarbodiimide/N-hydroxybenztriazole, dicyclohexylcarbodiimide/hydroxysuccinimide, 1-ethyl-3-(3-dimethylaminopropyl)carbodiimide.HCl/N-hydroxybenztriazole or 1-propanephosphonic acid cyclic anhydride.

As for the above 1) pathway, 5-amino-2,4,6-triiodo-3-chlorocarbonylbenzamide (C-IV) is coupled by malonic acid and phosphorus trichloride (PCl₃) in freshly distilled THF at 45-50° C. The product 3,3′-[(1,3-dioxo-1,3-propanediyl)diimino]bis[5-(aminocarbonyl)-2,4,6-triiodobenzoyl chloride (C-V) is filtered, washed by freshly distilled tetrahydrofuran and dried.

As for the above 2) pathway, 3-amino-5-(aminocarbonyl)-2,4,6-triiodobenzoyl chloride (C-IV) is coupled by malonic acid in presence of coupling agent 1-propanephosphonic acid cyclic anhydride.

Step 4

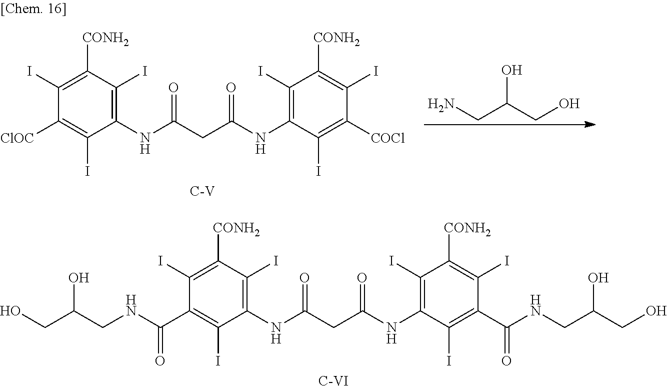

Improved process to intermediate C-VI (3,3′-[(1,3-dioxo-1,3-propanediyl)diimino]bis[5-(aminocarbonyl)-2,4,6-triiodobenzoyl chloride]) is based on amidation of C-V by 3-amino-propane-1,2-diol in N,N-dimethylformamide, at 2-25° C. The reaction mixture is purified by ionex resins and precipitated and crystallized in mixture of isopropanol-water. The product is filtered, washed with acetone and dried.

Step

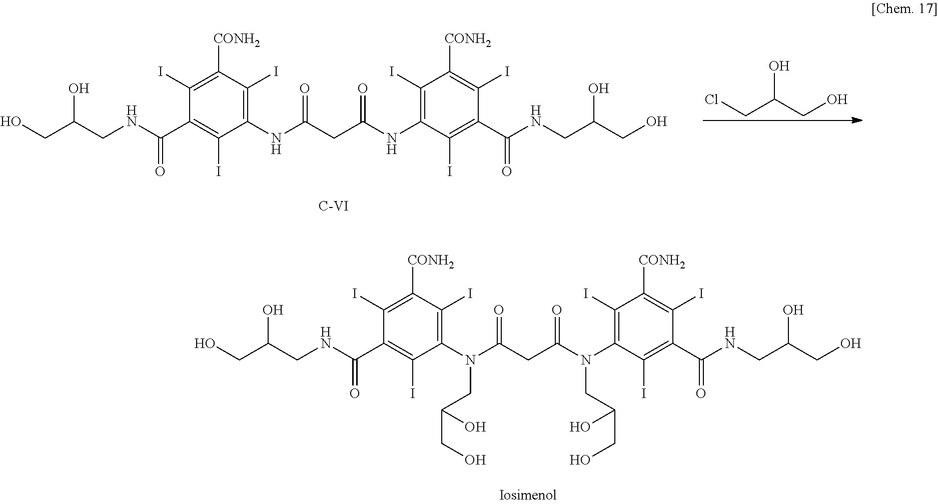 [SMILES: NC(=O)c1c(I)c(NC(=O)CC(=O)Nc2c(I)c(C(N)=O)c(I)c(C(=O)NCC(O)CO)c2I)c(I)c(C(=O)NCC(O)CO)c1I ; OCC(O)CCl ; "C-VI" ; NC(=O)c1c(I)c(C(=O)NCC(O)CO)c(I)c(N(CC(O)CO)C(=O)CC(=O)N(CC(O)CO)c2c(I)c(C(N)=O)c(I)c(C(=O)NCC(O)CO)c2I)c1I]

Crude iosimenol can be prepared by alkylation of C-VI with 3-chloro-propane-1,2-diol using non-aqueous organic solvent in presence of anhydrous calcium chloride and lithium hydroxide. This achievement was done through implementation of gradual addition of 3-chloro-propane-1,2-diol into reaction mixture and lowering of reaction temperature.

The process for production crude iosimenol can be done by use of various solvents like 2-methoxyethanol, dimethyl sulfoxide, N,N-dimethylformamide, N-methyl-2-pyrrolidone, ethylene glycol or propylene glycol. Lithium hydroxide, lithium hydroxide hydrate, calcium oxide or calcium hydroxide were used as base. Glycerol anhydrous was used for improving solubility of C-VI. Calcium chloride was used to reduce overalkylation reaction. Alkylation agent, 3-chloro-1,2-propandiol, was added gradually or in several portions.

Steps 6-8

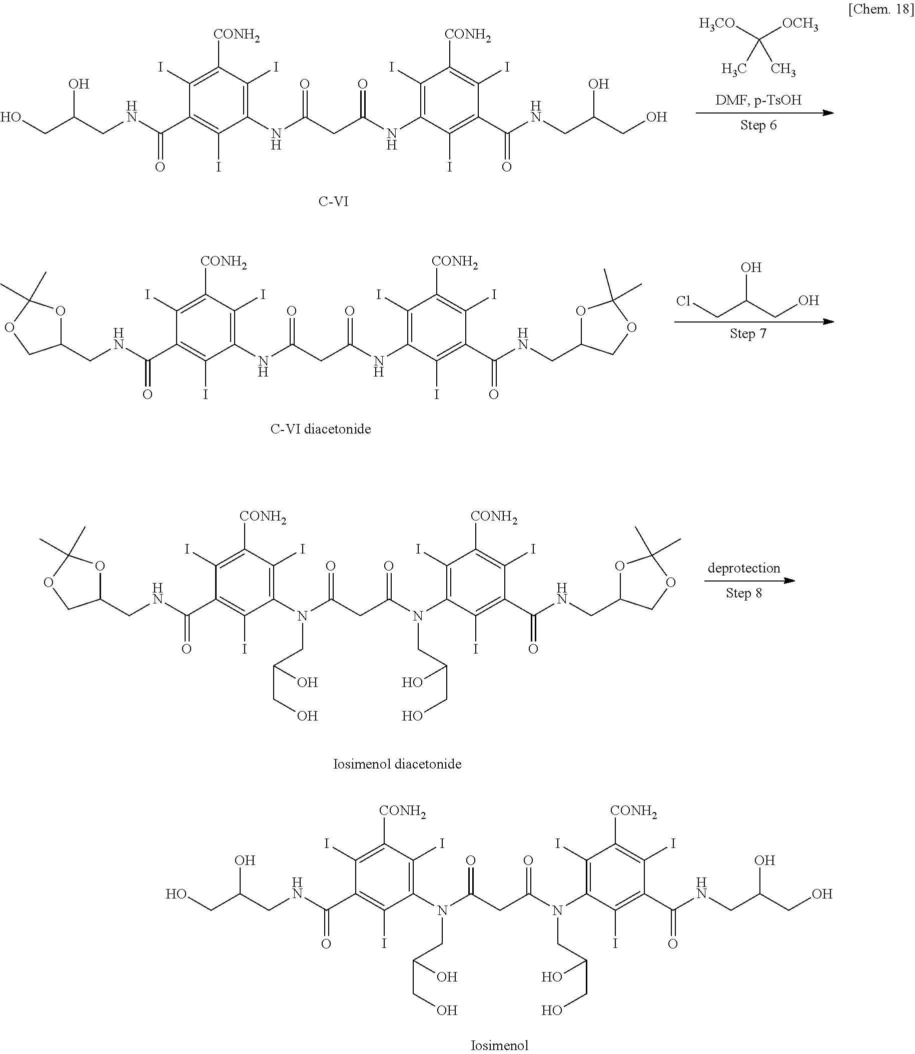

We developed the process for preparation of protected C-VI (hereinafter called “C-VI diacetonide”) using 2,2-dimethoxypropane as protective agent. The process is different from procedure described in U.S. Pat. No. 5,698,739. C-VI diacetonide (5,5′-(malonylbis(azanediyl))bis(N-((2,2-dimethyl-1,3-dioxolan-4-yl)methyl)-2,4,6-trii odoisophthalamide) reacts with 3-chloro-propane-1,2-diol in methanol in presence of lithium hydroxide monohydrate, anhydrous calcium chloride and glycerin at 10-60° C. Alternatively, synthesis of C-VI diacetonide starts from C-V as one-pot process, where no C-VI is isolated and reaction continues directly to obtain C-VI diacetonide.

Process of Purifying Iosimenol

For the purification of crude iosimenol, a crystallization is useful, and a preferred solvent for the crystallization is a solvent comprising 2-methoxyethanol for increasing its yield. More preferably, the solvent for the crystallization is a binary or tertiary solvent-mixture selected from 2-methoxyethanol, methoxy-2-propanol, and alcohols which a selected from methanol, ethanol, 2-propanol, n-butanol and/or 2-butanol.

Other preferred solvents for the crystallization include glycols such as diethylene glycol and triethylene glycol. 2-Ethoxyethanol or 1-methoxy-2-propanol are also useful as a solvent for the crystallization.

And also, the presence of water in the crystallization solvent is very important for the crystallization. The amount of water is preferably 1 v/v % to 10 v/v %.

In addition, for the crystallization, it is preferable to include tromethamol. The amount of tromethamol is preferably 0.1 w/v % to 0.5 w/v %.

### EXAMPLES

Hereinafter, the present invention is illustrated by the following examples, but should not be construed to be limited thereto, and it is possible to vary each condition unless the variation is beyond the range of the present invention.

### Example 1. Preparation of C-III (3-amino-5-(aminocarbonyl)-2,4,6-triiodobenzoic Acid

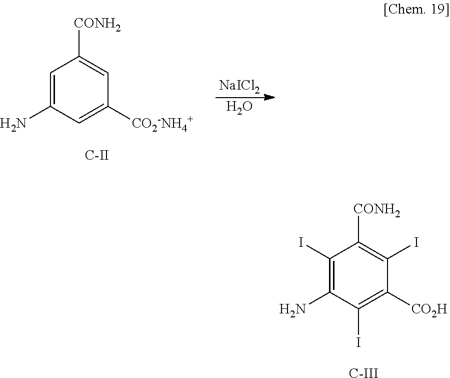

A 2500 L glass-lined reactor was charged with 5% wt. aqueous solution of the ammonium salt of 3-amino-5-(aminocarbonyl)benzoic acid (2020 kg, 512.2 moles) and 35% wt. hydrochloric acid (99 kg). The reaction mixture was heated to 60-70° C. and aqueous solution of sodium iodine dichloride of concentration 0.49 kg/L (534 L, 1611 moles) was added over 70-90 minutes. The reaction mixture was stirred at 75-80° C. for 2 hours. The reaction mixture was then cooled to 10-15° C. to provide a solid product. The solid product was washed with a 0.01% HCl solution (1000 L) then with a solution (1000 L) that contained sodium metabisulfite (1.5 kg) and finally with another 0.01% HCl solution (1000 L). The solid product was dissolved by adding sodium hydroxide (18.1 kg) in aqueous solution (940 L) and sodium carbonate (2.3 kg) was added to bring the pH to 10-11. The solution was warmed to 45-50° C. and active charcoal (8 kg) was added. The charcoal was removed by filtration and absolute ethanol (175 L) was added. The product was precipitated by adding 15% HCl (112 L) to pH 1.4-1.7. Crude 3-amino-5-(aminocarbonyl)-2,4,6-triiodobenzoic acid was collected by filtration and washed with demineralized water (200 L). The crude C-III dried product (226 kg, yield was 79% th), 3-amino-5-(aminocarbonyl)-2,4,6-triiodobenzoic acid (batch: 00103004), had a chromatographic purity of 98.7 area %.

### Example 2. Preparation of C-III Methanol Solvate (3-amino-5-(aminocarbonyl)-2,4,6-triiodobenzoic Acid Methanol Solvate)

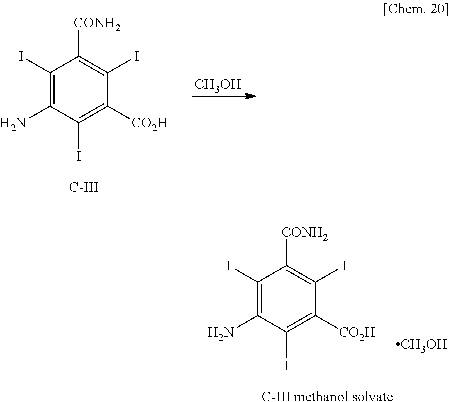

A 6000 mL three neck round-bottomed flask equipped with a magnetic stirrer was charged with the pre-purified intermediate C-III (batch: 00103004, 500 g) and methanol (2000 mL). The stirred suspension was heated up to reflux and maintain for 2-3 hours. Then, the suspension was cooled down and agitated for two hours at 5-10° C. Precipitate of C-III was filtered off and without drying continues into next step.

In the second step, all wet precipitate of C-III was suspended in 2000 mL of methanol in 6000 mL three neck round-bottomed flasks equipped with a magnetic stirrer. The stirred suspension was heated up and agitated under reflux for 2-3 hours. Then, the suspension was cooled down to 5-10° C., solid C-III was filtered off and dried under vacuum for 48 hours at 58-60° C. to yield 3-amino-5-(aminocarbonyl)-2,4,6-triiodobenzoic acid methanol solvate (C-III methanol solvate) as a white powder (466.5 grams, yield of purification 93.3% (yield calculated on starting C-III crude), HPLC purity of 99.59% Area, lot number: JM120716A). In a similar manner to the above procedure, some working examples were prepared, the results of which are shown in Table 1.

TABLE 1

| | | Amount of | | Purification | | |
|---|---|---|---|---|---|---|
| | Amount of | used solvent | | step | | |
| | starting | in | | (Number of | | |
| Batch | material | MeOH | water | Re-slurry | C-III purity | |
| Number | batch (g) | (mL) | (mL) | steps) | (% HPLC area) | Yield: |
| JM120516A | 200 | 1000 | — | 3x | 99.78% | 81.8% |
| JM120516B | 100 | 1180 | 100 | 3x | 99.87% | 57.1% |
| JM180516 | 1000 | 1100 | — | 3x | 99.71% | 85.1% |
| JM200616A | 500 | 2000 | 10 | 3x | 99.67% | 88.4% |
| JM200616B | 500 | 2000 | 10 | 3x with 1 g | 99.73% | 84.0% |
| | | | | of Tris | | |
| JM120716A | 500 | 2000 | — | 2x | 99.59% | 93.3% |
| JM120716B | 500 | 2000 | — | 2x | 99.65% | 93.9% |

### Example 3. Purification of C-III Methanol Solvate (3-amino-5-(aminocarbonyl)-2,4,6-triiodobenzoic Acid Methanol Solvate)

For purpose of testing, the title compound 3-amino-5-carbamoyl-2,4,6-triiodobenzoic acid methanol solvate (C-III methanol solvate) was purified according to the following process.

Pre-purified C-III (10 g) is suspended in methanol (150 mL) and water (10 mL). It is stirred under reflux for 8-20 hours. Cooled reaction mixture is filtered through a S4 frit. Mother liquor is concentrated on rotary evaporator and left in refrigerator overnight. Off-white solid is collected and dried overnight at 60° C. Combined solids (at least 62% yields) are recrystallized again with methanol (150 mL) and water (10 mL). Collected solid is dried overnight at 60° C.

(Analyses)

Density Measurement

For the title compound, experimental density was evaluated as an average of ten measurements in 50 ml pycnometer, using n-heptane as a liquid, resulting in a value of 2.666±0.031 g/cm³. Computed density is 2,641 g/cm³. Deviation from experimental value is below 1% and lies within the range of standard error.

Melting Point Measurement

Capillary melting point apparatus Electrothermal IA9200 was used for the evaluation of melting point. Sample was heated from room temperature to 250° C. at the rate of 10° C./min and then at 0.2° C./min. It is observable in the capillary apparatus that release of iodine vapors occurs at 230° C. and the specimen decomposes prior its melting point. At 300° C., no solid particles were observed.

Thermogravimetric Analyses

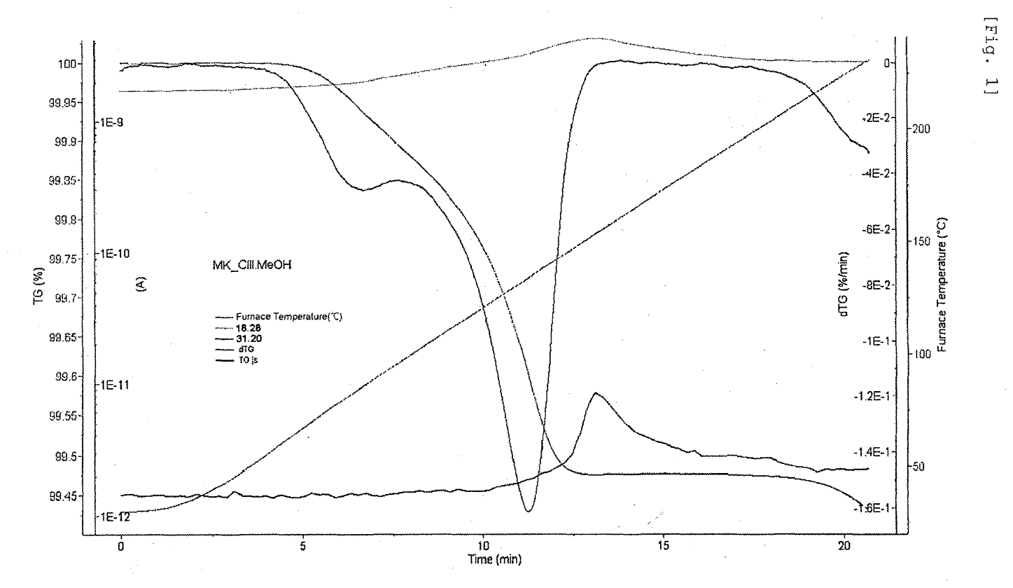

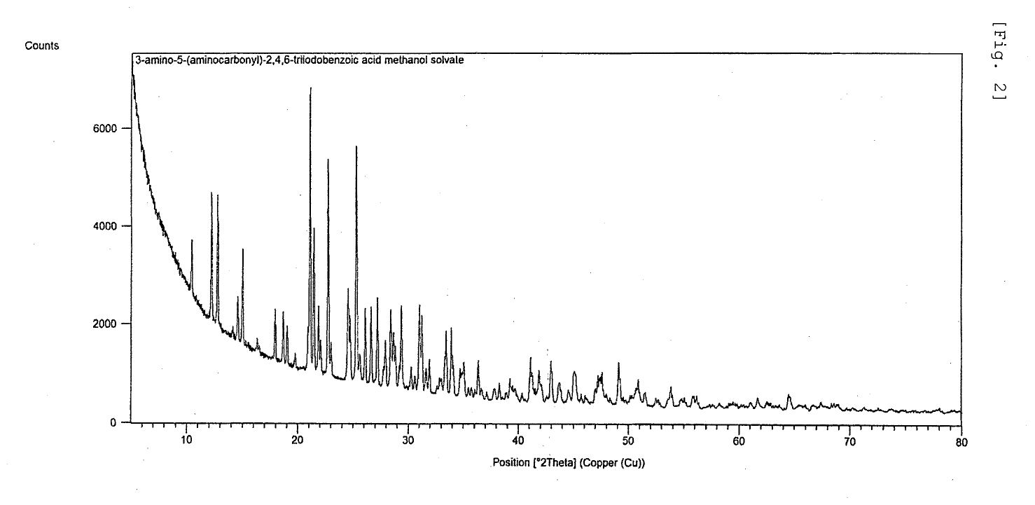

The aforementioned findings during melting point measurement were further confirmed by TGA, on Setsys Evolution 1750 (Seratam) coupled with MS Omnistar (Pfiffer). C-III sample (28,143 mg) was placed in platinum crucible at 30° C. and cell was flushed with helium (2 hours at 60 ml/min). Heating ramp was set as 10° C./min. MS evaluation of gases released upon heating showed methanol being released from the sample at 120° C. and primarily at 217° C. However, this was accompanied by water, thus making the sample to be quite hygroscopic. At 230° C. TGA was stopped due to the iodine vapors being released by the decomposed molecule. The mass loss was only 0.313%, accounted for methanol and water together. This method is not suitable for determination of various sample properties based on higher warmth needed to be applied, e.g. calculation of C-III:MeOH ratio (this was computed by NMR). The overview graph of thermogravimetric analysis of C-III methanol solvate is shown in FIG. 1.

XRD Measurement

Sample was prepared by careful grinding in an agate mortar and front-loaded into the specimen holder. The diffraction pattern for the title compound was collected at room temperature with an X'Pert³ Powder θ-θ powder diffractometer with parafocusing Bragg-Brentano geometry using Cu Kα radiation (λ=1.5418 Å, Ni filter, generator setting: 40 kV, 30 mA). An ultrafast PIXCEL detector was employed to collect XRD data over the angular range from 5 to 80° 2θ with a step size of 0.013° 2θ and a counting time of 118.32 s/step.

The software package HighScore Plus V 3.0e (PANalytical, Almelo, Netherlands) was used to smooth the data, to fit the background, to eliminate the Kα₂ component and the top of the smoothed peaks were used to determine the peak positions and intensities of the diffraction peaks (Table 1). The d-values were calculated using Cu Kα₁ radiation (λ=1.5406 Å).

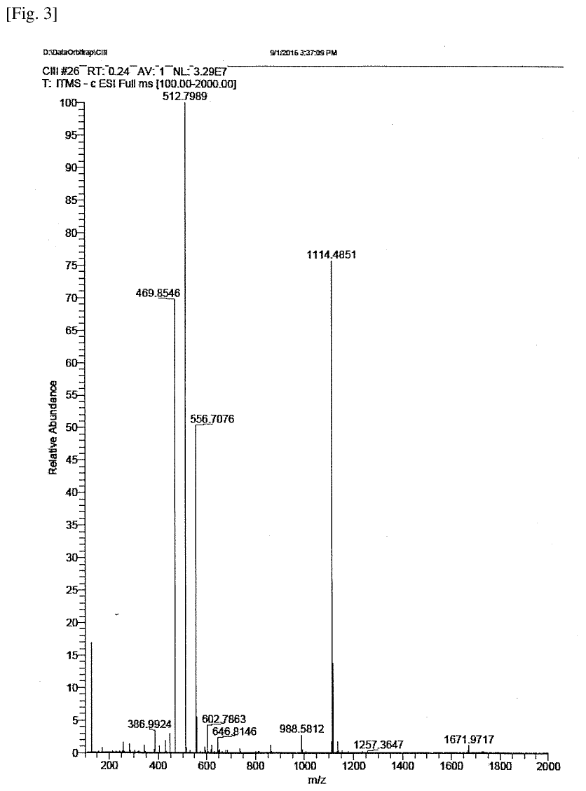

The experimental powder diffraction pattern is depicted in FIG. 2. Automatic indexing of results obtained using DICVOL04 show that title compound C₉H₉I₃N₂O₄ is orthorombic with space group Pbca and unit-cell parameters: a=17.000 (1) Å, b=13.896 (1) Å, c=12.597 (1) Å, unit-cell volume V=2975.9 Å³, M=589.89 g/mol, T_{m.p}. (melting point)=300(1) ° C., r_{c}=2.641 g·cm³, rₘ=2.66(3) g·cm³ and Z=8. The figures of merits are F₂₀=70.5(0.0053,53) and M₂₀=28.7. All measured lines were indexed and are consistent with the Pbca space group. No detectable impurities were observed. Detailed data are provided in Table 2.

Indexed X-ray powder diffraction data for C₉H₉I₃N₂O₄. Only the peaks with Iᵣₑₗ of 1 or greater are presented presented [a=17.000 (1) Å, b=13.896 (1) Å, c=12.597 (1) Å, unit-cell volume V=2975.9 Å³, Z=8, space group Pbca]. All lines were indexed and are consistent with the Pbca space group. The d-values were calculated using Cu Kα₁ radiation (λ=1.5406 Å).

TABLE 2

| 2q_{obs} (°) | d_{obs} (Å) | I_{obs} | h | k | l | 2q_{cal} (°) | d_{cal} (Å) | Δ2q |
|---|---|---|---|---|---|---|---|---|
| 10.438 | 8.4683 | 19 | 2 | 0 | 0 | 10.399 | 8.5002 | −0.039 |
| 12.228 | 7.2323 | 45 | 2 | 1 | 0 | 12.196 | 7.2512 | −0.032 |
| 12.769 | 6.9269 | 46 | 0 | 2 | 0 | 12.730 | 6.9481 | −0.039 |
| 14.126 | 6.2645 | 3 | 2 | 1 | 1 | 14.081 | 6.2843 | −0.045 |
| 14.582 | 6.0699 | 15 | 0 | 2 | 1 | 14.548 | 6.0840 | −0.034 |
| 15.020 | 5.8936 | 34 | 1 | 0 | 2 | 14.989 | 3.9060 | −0.032 |
| 16.324 | 5.4256 | 5 | 1 | 1 | 2 | 16.295 | 5.4355 | −0.030 |
| 16.500 | 5.3683 | 3 | 2 | 2 | 0 | 16.465 | 5.3796 | −0.035 |
| 17.947 | 4.9385 | 18 | 2 | 2 | 1 | 17.915 | 4.9473 | −0.032 |
| 18.673 | 4.7482 | 18 | 2 | 1 | 2 | 18.616 | 4.7550 | −0.027 |
| 19.035 | 4.6586 | 13 | 0 | 2 | 2 | 19.003 | 4.6664 | −0.032 |
| 19.742 | 4.4934 | 4 | 1 | 2 | 2 | 19.713 | 4.5000 | −0.029 |
| 20.915 | 4.2440 | 15 | 4 | 0 | 0 | 20.884 | 4.2501 | −0.030 |
| 21.097 | 4.2077 | 100 | 1 | 3 | 1 | 21.076 | 4.2119 | −0.021 |
| 21.438 | 4.1417 | 50 | 3 | 2 | 1 | 21.411 | 4.1467 | −0.026 |
| 21.867 | 4.0612 | 24 | 4 | 1 | 0 | 21.851 | 4.0643 | −0.017 |
| 22.051 | 4.0278 | 12 | 3 | 1 | 2 | 22.031 | 4.0315 | −0.020 |
| 22.739 | 3.9074 | 76 | 1 | 1 | 3 | 22.715 | 3.9115 | −0.024 |
| 22.991 | 3.8652 | 12 | 4 | 1 | 1 | 22.975 | 3.8679 | −0.017 |
| 24.551 | 3.6230 | 32 | 4 | 2 | 0 | 24.533 | 3.6236 | −0.018 |
| 24.716 | 3.5992 | 22 | 3 | 2 | 2 | 24.695 | 3.6023 | −0.022 |
| 25.285 | 3.5195 | 83 | 4 | 0 | 2 | 25.259 | 3.5230 | −0.026 |
| 25.578 | 3.4799 | 10 | 4 | 2 | 1 | 25.546 | 3.4841 | −0.032 |
| 25.637 | 3.4719 | 9 | 0 | 4 | 0 | 25.621 | 3.4741 | −0.016 |
| 26.083 | 3.4135 | 26 | 4 | 1 | 2 | 26.072 | 3.4150 | −0.011 |
| 26.628 | 3.3450 | 27 | 0 | 4 | 1 | 26.595 | 3.3490 | −0.033 |
| 27.194 | 3.2766 | 31 | 3 | 1 | 3 | 27.178 | 3.2784 | −0.016 |
| 27.752 | 3.2120 | 7 | 2 | 4 | 0 | 27.718 | 3.2158 | −0.034 |
| 27.922 | 3.1928 | 16 | 5 | 1 | 1 | 27.905 | 3.1947 | −0.016 |
| 28.402 | 3.1399 | 27 | 4 | 2 | 2 | 28.381 | 3.1422 | −0.021 |
| 28.651 | 3.1132 | 19 | 2 | 4 | 1 | 28.626 | 3.1159 | −0.026 |
| 28.826 | 3.0947 | 15 | 1 | 0 | 4 | 28.809 | 3.0965 | −0.017 |
| 29.183 | 3.0577 | 8 | 1 | 3 | 3 | 29.159 | 3.0601 | −0.024 |
| 29.366 | 3.0391 | 29 | 4 | 3 | 1 | 29.365 | 3.0391 | −0.001 |
| 29.847 | 2.9911 | 1 | 5 | 0 | 2 | 29.839 | 2.9919 | −0.008 |
| 30.080 | 2.9684 | 1 | 5 | 2 | 1 | 30.085 | 2.9680 | 0.004 |
| 30.251 | 2.9521 | 8 | 2 | 0 | 4 | 30.241 | 2.9530 | −0.010 |
| 30.594 | 2.9198 | 5 | 4 | 1 | 3 | 30.388 | 2.9203 | −0.006 |
| 31.016 | 2.8810 | 30 | 3 | 4 | 1 | 30.992 | 2.8832 | −0.024 |
| 31.224 | 2.8623 | 27 | 2 | 4 | 2 | 31.204 | 2.8641 | −0.020 |
| 31.616 | 2.8277 | 8 | 1 | 2 | 4 | 31.609 | 2.8283 | −0.007 |
| 31.903 | 2.8029 | 12 | 4 | 3 | 2 | 31.889 | 2.8041 | −0.015 |
| 32.600 | 2.7445 | 2 | 4 | 2 | 3 | 32.605 | 2.7442 | 0.004 |
| 32.832 | 2.7257 | 6 | 3 | 3 | 3 | 32.815 | 2.7270 | −0.017 |
| 32.926 | 2.7181 | 5 | 2 | 2 | 4 | 32.931 | 2.7177 | 0.005 |
| 33.012 | 2.7112 | 5 | 6 | 1 | 1 | 33.012 | 2.7112 | 0.000 |
| 33.313 | 2.6874 | 13 | 4 | 4 | 0 | 33.283 | 2.6898 | −0.030 |
| 33.425 | 2.6786 | 22 | 5 | 3 | 1 | 33.430 | 2.6783 | 0.005 |
| 33.895 | 2.6426 | 23 | 2 | 5 | 0 | 33.908 | 2.6416 | 0.013 |
| 34.073 | 2.6292 | 10 | 4 | 4 | 1 | 34.056 | 2.6305 | −0.018 |
| 34.696 | 2.5834 | 10 | 6 | 0 | 2 | 34.688 | 2.5840 | −0.008 |
| 34.816 | 2.5748 | 8 | 1 | 3 | 4 | 34.823 | 2.5743 | 0.007 |
| 34.913 | 2.5679 | 8 | 6 | 2 | 1 | 34.903 | 2.5685 | −0.009 |
| 35.039 | 2.5589 | 12 | 3 | 2 | 4 | 35.035 | 2.5591 | −0.004 |
| 35.454 | 2.5299 | 3 | 4 | 0 | 4 | 35.448 | 2.5303 | −0.006 |
| 35.731 | 2.5109 | 3 | 4 | 3 | 3 | 35.739 | 2.5103 | 0.008 |
| 36.054 | 2.4891 | 3 | 4 | 1 | 4 | 36.051 | 2.4893 | −0.003 |
| 36.349 | 2.4696 | 13 | 5 | 2 | 3 | 36.346 | 2.4698 | −0.003 |
| 36.637 | 2.4508 | 3 | 1 | 1 | 5 | 36.604 | 2.4530 | −0.033 |
| 36.686 | 2.4477 | 3 | 3 | 5 | 1 | 36.685 | 2.4477 | −0.001 |
| 37.126 | 2.4197 | 2 | 3 | 4 | 3 | 37.117 | 2.4203 | −0.009 |
| 37.704 | 2.3839 | 1 | 5 | 4 | 1 | 37.670 | 2.3860 | −0.034 |
| 37.803 | 2.3779 | 3 | 4 | 2 | 4 | 37.809 | 2.3775 | 0.007 |
| 37.862 | 2.3743 | 3 | 6 | 3 | 1 | 37.871 | 2.3738 | 0.009 |
| 37.959 | 2.3685 | 10 | 0 | 2 | 5 | 37.960 | 2.3684 | 0.001 |
| 38.270 | 2.3499 | 6 | 7 | 1 | 1 | 38.263 | 2.3503 | −0.007 |
| 38.364 | 2.3444 | 3 | 1 | 2 | 5 | 38.341 | 2.3458 | −0.024 |
| 38.862 | 2.3155 | 2 | 6 | 1 | 3 | 38.856 | 2.3158 | −0.006 |
| 38.954 | 2.3102 | 3 | 5 | 0 | 4 | 38.951 | 2.3104 | −0.003 |
| 39.232 | 2.2945 | 7 | 5 | 3 | 3 | 39.220 | 2.2952 | −0.013 |
| 39.341 | 2.2884 | 5 | 4 | 5 | 1 | 39.359 | 2.2874 | 0.018 |
| 39.473 | 2.2811 | 5 | 2 | 2 | 5 | 39.465 | 2.2815 | −0.008 |
| 39.652 | 2.2712 | 4 | 3 | 1 | 5 | 39.653 | 2.2711 | 0.001 |
| 39.743 | 2.2662 | 4 | 7 | 0 | 2 | 39.746 | 2.2660 | 0.004 |
| 39.907 | 2.2572 | 2 | 1 | 6 | 1 | 39.899 | 2.2577 | −0.009 |
| 40.333 | 2.2344 | 3 | 2 | 6 | 0 | 40.329 | 2.2346 | −0.004 |
| 41.096 | 2.1946 | 16 | 1 | 3 | 5 | 41.096 | 2.1946 | 0.000 |
| 41.289 | 2.1848 | 9 | 3 | 2 | 5 | 41.281 | 2.1852 | −0.009 |
| 41.521 | 2.1731 | 5 | 0 | 6 | 2 | 41.509 | 2.1737 | −0.012 |
| 41.758 | 2.1614 | 4 | 6 | 4 | 1 | 41.723 | 2.1631 | −0.035 |
| 41.877 | 2.1555 | 11 | 1 | 6 | 2 | 41.863 | 2.1562 | −0.014 |
| 42.099 | 2.1446 | 6 | 3 | 5 | 3 | 42.090 | 2.1451 | −0.010 |
| 42.582 | 2.1214 | 2 | 5 | 5 | 1 | 42.589 | 2.1211 | 0.007 |
| 42.947 | 2.1042 | 14 | 5 | 4 | 3 | 42.970 | 2.1032 | 0.023 |
| 43.046 | 2.0996 | 10 | 0 | 0 | 6 | 43.050 | 2.0994 | 0.004 |
| 43.638 | 2.0725 | 6 | 8 | 1 | 1 | 43.649 | 2.0720 | 0.011 |
| 43.732 | 2.0683 | 7 | 1 | 5 | 4 | 43.731 | 2.0683 | −0.001 |
| 43.880 | 2.0616 | 4 | 3 | 3 | 5 | 43.883 | 2.0615 | 0.003 |
| 44.544 | 2.0324 | 5 | 8 | 2 | 0 | 44.551 | 2.0321 | 0.007 |
| 44.642 | 2.0282 | 4 | 0 | 6 | 3 | 44.647 | 2.0280 | 0.005 |
| 45.005 | 2.0127 | 10 | 7 | 2 | 3 | 45.016 | 2.0122 | 0.012 |
| 45.081 | 2.0095 | 11 | 0 | 2 | 6 | 45.076 | 2.0097 | −0.005 |
| 45.223 | 2.0035 | 10 | 5 | 1 | 5 | 45.233 | 2.0031 | 0.010 |
| 45.715 | 1.9831 | 4 | 2 | 4 | 5 | 45.711 | 1.9832 | −0.004 |
| 46.067 | 1.9687 | 3 | 3 | 0 | 6 | 46.068 | 1.9687 | 0.002 |
| 46.322 | 1.9585 | 2 | 6 | 5 | 1 | 46.286 | 1.9599 | −0.036 |
| 46.982 | 1.9325 | 5 | 2 | 7 | 0 | 46.965 | 1.9332 | −0.018 |
| 47.217 | 1.9234 | 10 | 7 | 0 | 4 | 47.224 | 1.9232 | 0.007 |
| 47.350 | 1.9183 | 9 | 3 | 4 | 5 | 47.333 | 1.9190 | −0.017 |
| 47.450 | 1.9145 | 10 | 7 | 3 | 3 | 47.454 | 1.9144 | 0.004 |
| 47.587 | 1.9093 | 12 | 3 | 6 | 3 | 47.585 | 1.9094 | −0.002 |
| 47.996 | 1.8940 | 4 | 3 | 2 | 6 | 47.993 | 1.8941 | −0.003 |
| 48.307 | 1.8825 | 3 | 4 | 0 | 6 | 48.313 | 1.8823 | 0.006 |
| 49.084 | 1.8545 | 15 | 1 | 6 | 4 | 49.081 | 1.8546 | −0.003 |
| 49.532 | 1.8388 | 3 | 4 | 4 | 5 | 49.534 | 1.8387 | 0.001 |
| 49.761 | 1.8309 | 2 | 4 | 6 | 3 | 49.778 | 1.8303 | 0.016 |
| 50.156 | 1.8174 | 4 | 6 | 2 | 5 | 50.162 | 1.8172 | 0.006 |
| 50.303 | 1.8124 | 4 | 8 | 4 | 0 | 50.292 | 1.8128 | −0.011 |
| 50.544 | 1.8044 | 4 | 9 | 2 | 1 | 50.555 | 1.8040 | 0.011 |
| 50.705 | 1.7990 | 6 | 4 | 7 | 0 | 50.715 | 1.7986 | 0.010 |
| 50.872 | 1.7935 | 9 | 6 | 6 | 0 | 50.880 | 1.7932 | 0.008 |
| 51.421 | 1.7756 | 4 | 6 | 6 | 1 | 51.430 | 1.7753 | 0.009 |
| 51.506 | 1.7729 | 5 | 3 | 5 | 5 | 51.506 | 1.7729 | 0.000 |
| 52.478 | 1.7423 | 3 | 8 | 4 | 2 | 52.485 | 1.7421 | 0.008 |
| 52.703 | 1.7354 | 3 | 7 | 1 | 5 | 52.722 | 1.7348 | 0.019 |
| 53.533 | 1.7104 | 3 | 3 | 7 | 3 | 53.515 | 1.7109 | −0.017 |
| 53.843 | 1.7013 | 7 | 2 | 8 | 0 | 53.824 | 1.7019 | −0.019 |
| 54.703 | 1.6766 | 3 | 7 | 5 | 3 | 54.700 | 1.6767 | −0.003 |
| 54.814 | 1.6734 | 3 | 8 | 5 | 1 | 54.824 | 1.6732 | 0.010 |
| 55.068 | 1.6663 | 4 | 1 | 8 | 2 | 55.063 | 1.6665 | −0.004 |
| 55.812 | 1.6459 | 4 | 2 | 3 | 7 | 55.820 | 1.6456 | 0.008 |
| 55.939 | 1.6424 | 4 | 2 | 8 | 2 | 55.920 | 1.6429 | −0.019 |
| 56.091 | 1.6383 | 3 | 6 | 2 | 6 | 56.058 | 1.6392 | −0.033 |
| 56.186 | 1.6358 | 4 | 7 | 3 | 5 | 56.185 | 1.6358 | −0.001 |
| 57.356 | 1.6052 | 2 | 0 | 8 | 3 | 57.359 | 1.6051 | 0.002 |
| 57.657 | 1.5975 | 1 | 10 | 2 | 2 | 57.664 | 1.5973 | 0.007 |
| 58.246 | 1.5827 | 2 | 4 | 6 | 5 | 58.258 | 1.5824 | 0.012 |
| 58.919 | 1.5663 | 1 | 8 | 5 | 3 | 58.918 | 1.5663 | −0.001 |
| 59.103 | 1.5618 | 2 | 7 | 4 | 5 | 59.103 | 1.5618 | 0.000 |
| 59.268 | 1.5579 | 2 | 4 | 8 | 2 | 59.264 | 1.5580 | −0.004 |
| 59.463 | 1.5532 | 2 | 1 | 7 | 5 | 59.483 | 1.5527 | 0.020 |
| 59.648 | 1.5489 | 2 | 1 | 6 | 6 | 59.641 | 1.5490 | −0.006 |
| 59.846 | 1.5442 | 2 | 3 | 8 | 3 | 59.839 | 1.5443 | −0.007 |
| 60.214 | 1.5356 | 2 | 0 | 2 | 8 | 60.214 | 1.5356 | 0.000 |
| 66.432 | 1.5306 | 2 | 2 | 6 | 6 | 60.456 | 1.5301 | 0.024 |
| 60.667 | 1.5253 | 1 | 7 | 7 | 1 | 60.647 | 1.5257 | −0.020 |
| 61.023 | 1.5172 | 2 | 3 | 0 | 8 | 61.028 | 1.5171 | 0.005 |
| 61.127 | 1.5149 | 2 | 1 | 8 | 4 | 61.124 | 1.5149 | −0.003 |
| 61.700 | 1.5022 | 4 | 5 | 8 | 2 | 61.698 | 1.5022 | −0.003 |
| 62.526 | 1.4843 | 3 | 10 | 4 | 2 | 62.539 | 1.4840 | 0.013 |
| 62.693 | 1.4807 | 2 | 6 | 8 | 0 | 62.686 | 1.4809 | −0.007 |
| 62.885 | 1.4767 | 2 | 9 | 2 | 5 | 62.881 | 1.4768 | −0.005 |
| 63.296 | 1.4681 | 2 | 9 | 4 | 4 | 63.294 | 1.4681 | −0.002 |
| 63.653 | 1.4607 | 2 | 4 | 6 | 6 | 63.652 | 1.4607 | −0.001 |
| 64.100 | 1.4516 | 1 | 5 | 8 | 3 | 64.105 | 1.4515 | 0.004 |
| 64.480 | 1.4440 | 5 | 1 | 9 | 3 | 64.482 | 1.4439 | 0.002 |
| 64.625 | 1.4411 | 5 | 8 | 7 | 1 | 64.621 | 1.4411 | −0.003 |
| 65.252 | 1.4287 | 1 | 5 | 0 | 8 | 65.248 | 1.4288 | −0.004 |
| 65.404 | 1.4258 | 2 | 9 | 6 | 2 | 65.402 | 2.4258 | −0.002 |
| 65.517 | 1.4236 | 2 | 10 | 3 | 4 | 65.518 | 1.4236 | 0.001 |
| 65.604 | 1.4219 | 2 | 2 | 7 | 6 | 65.594 | 1.4221 | −0.011 |
| 65.717 | 1.4197 | 2 | 11 | 2 | 3 | 65.716 | 1.4198 | −0.002 |
| 65.883 | 1.4166 | 2 | 12 | 0 | 0 | 65.876 | 1.4167 | −0.008 |
| 66.006 | 1.4142 | 2 | 4 | 9 | 2 | 66.008 | 1.4142 | 0.002 |
| 66.571 | 1.4036 | 2 | 11 | 4 | 1 | 66.587 | 1.4033 | 0.017 |
| 66.672 | 1.4017 | 2 | 2 | 6 | 7 | 66.680 | 1.4016 | 0.007 |
| 66.944 | 1.3967 | 2 | 6 | 8 | 3 | 66.948 | 1.3966 | 0.004 |
| 67.200 | 1.3920 | 2 | 6 | 4 | 7 | 67.208 | 1.3918 | 0.008 |
| 67.400 | 1.3883 | 3 | 5 | 8 | 4 | 67.395 | 1.3884 | −0.005 |
| 67.730 | 1.3823 | 2 | 9 | 6 | 3 | 67.737 | 1.3822 | 0.007 |
| 68.353 | 1.3713 | 3 | 8 | 7 | 3 | 68.360 | 1.3711 | 0.007 |
| 68.569 | 1.3675 | 3 | 1 | 2 | 9 | 68.561 | 1.3676 | −0.008 |
| 68.797 | 1.3635 | 3 | 6 | 6 | 6 | 68.795 | 1.3635 | −0.002 |
| 68.945 | 1.3609 | 2 | 11 | 2 | 4 | 68.967 | 1.3606 | 0.021 |
| 69.698 | 1.3481 | 1 | 6 | 9 | 1 | 69.701 | 1.3480 | 0.004 |
| 70.227 | 1.3392 | 1 | 10 | 6 | 2 | 70.231 | 1.3391 | 0.004 |
| 71.184 | 1.3235 | 1 | 2 | 3 | 9 | 71.188 | 1.3235 | 0.004 |
| 71.261 | 1.3223 | 2 | 2 | 8 | 6 | 71.275 | 1.3220 | 0.014 |
| 71.914 | 1.3119 | 1 | 12 | 4 | 0 | 71.917 | 1.3118 | 0.003 |
| 72.296 | 1.3059 | 1 | 10 | 4 | 5 | 72.297 | 2.3059 | 0.001 |
| 72.529 | 1.3023 | 1 | 0 | 6 | 8 | 72.536 | 1.3021 | 0.007 |
| 73.530 | 1.2870 | 1 | 2 | 6 | 8 | 73.520 | 1.2871 | −0.011 |
| 73.718 | 1.2842 | 1 | 12 | 4 | 2 | 73.711 | 1.2843 | −0.006 |
| 74.028 | 1.2796 | 1 | 6 | 4 | 8 | 74.026 | 1.2796 | −0.001 |
| 74.691 | 1.2698 | 1 | 12 | 2 | 4 | 74.664 | 1.2702 | −0.027 |
| 74.966 | 1.2658 | 1 | 9 | 6 | 5 | 74.978 | 1.2657 | 0.012 |
| 75.346 | 1.2604 | 1 | 5 | 10 | 2 | 75.352 | 1.2603 | 0.006 |
| 75.595 | 1.2569 | 1 | 10 | 5 | 5 | 75.595 | 1.2569 | 0.000 |
| 75.846 | 1.2533 | 1 | 9 | 5 | 6 | 75.846 | 1.2533 | 0.001 |
| 76.427 | 1.2452 | 1 | 6 | 9 | 4 | 76.424 | 1.2453 | −0.003 |
| 76.756 | 1.2407 | 1 | 1 | 9 | 6 | 76.770 | 1.2405 | 0.014 |
| 77.474 | 1.2310 | 1 | 10 | 1 | 7 | 77.481 | 1.2309 | 0.007 |
| 77.746 | 1.2274 | 1 | 3 | 11 | 1 | 77.762 | 1.2272 | 0.017 |
| 78.008 | 1.2239 | 2 | 6 | 10 | 2 | 78.011 | 1.2239 | 0.003 |
| 79.068 | 1.2101 | 1 | 6 | 8 | 6 | 79.069 | 1.2101 | 0.001 |
| 79.336 | 1.2067 | 1 | 1 | 11 | 3 | 79.340 | 1.2067 | 0.004 |
| 79.760 | 1.2014 | 1 | 13 | 4 | 2 | 79.757 | 1.2014 | −0.003 |

Mass spectrometry

TABLE A
(Experimental conditions)

| Instrument: | LTQ Orbitrap Velos |
|---|---|
| Ionization: | ESI⁻ |
| Scan mode: | Full scan - 100-1000 Da |
| Direct injection: | Flow 2 mL/min of the sample |
| Spray Voltage: | −3000 V |
| Vaporizer Temperature: | 40° C. |
| Sheath Gas Pressure: | 10 psi |
| Aux Gas Pressure: | 5 psi |
| Capillary Temperature: | 270° C. |
| S-Lens RF Amplitude | 227 |

(Sample Preparation)

The sample (1 mg) was dissolved in water (1 mL). The result is shown in FIG. 3.

The mass spectrum of C-III was obtained by flow injection analysis in ESI⁺ and ESI⁻ ionization on LTQ Orbitrap Velos spectrometer. C—III contains molecular peak in ESI spectra m/z=556.7076 Da in negative ionization mode which corresponds to the [M-H]⁻ ion of the proposed structure, m/z=512.7989 Da in negative ionization mode which corresponds to the loss of [M-COO]⁻ ion of the proposed structure, and m/z=469.8546 Da in negative ionization mode which corresponds to the [M-COO—CONH]⁻ ion of the proposed structure. The last peak in the spectrum is double charged molecular ion of the proposed structure m/z=1114.4851 Da.

Nuclear Magnetic Resonance

NMR spectra were measured on a Bruker Avance III 600 (600.23 MHz for ¹H, 150.93 MHz for ¹³C, 60.82 MHz for ¹⁵N) in DMSO-d₆ at 30° C. The spectra were recorded using standard manufacturer's software Topspin 3.5 (Bruker BioSpin GmbH, Rheinstetten, Germany).

The following experiments were performed: ¹H NMR, ¹³C NMR, COSY, ¹H-¹³C HSQC, ¹H-¹³C HMBC, ¹H-¹⁵N HSQC.

TABLE 3

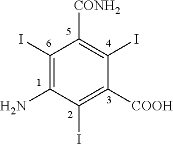 [SMILES: NC(=O)c1c(I)c(N)c(I)c(C(=O)O)c1I]

| | | | | | | | |
|---|---|---|---|---|---|---|---|
| | | | | | | J_{HH} | HMBC |
| Atom# | δ_{C} | m | δ_{N} | δ_{H} | m | [Hz] | (H to C) |

C-III

| | | | | | | | |
|---|---|---|---|---|---|---|---|
| 1, 3 | 147.98 | s | | — | — | — | — |
| | 147.50 | s | | | | | |
| 2, 6 | 77.35 | s | | — | — | — | 1-NH_{₂} |
| | 79.54 | s | | | | | |
| 4 | 71.50 | s | | — | — | — | — |
| 5 | 149.74 | s | | — | — | — | 5-CONH₂-u, |
| 5-C═O | 171.23 | s | | — | — | — | 5-CONH₂-u, |
| | | | | | | | 5-CONH₂-d |
| 3-C═O | 169.89 | s | | — | — | — | — |
| 5-CONH₂ | — | — | 110 | 7.834 | br d | 1.4 | |
| | | | | 7.570 | br d | 1.4 | |
| 1-NH₂ | — | — | 90 | 5.485 | s | — | |
| 3-COOH | — | — | | 13.659 | br.s | — | |

Methanol

| | | | | | |
|---|---|---|---|---|---|
| CH₃ | 48.50 | q | 3.169 | s | — |
| OH | — | | 4.058 | br s | — |

The ¹H NMR spectrum of the sample contains one one-proton singlet at 13.659 and one two-proton singlet 5.485 ppm and an AB spin system of weakly coupled protons attached to common nitrogen. Moreover, it was detected one methyl singlet of a methoxyl group, which is weakly coupled with broad singlet of hydrogen attached to a heteroatom in the ¹H NMR spectrum.
The ¹³C NMR spectrum contains eight quaternary carbons signals. Three up-field resonating carbons (79.54, 77.35, 71.50 ppm) are consistent with iodine substitution of benzene ring. Furthermore, it was detected three aromatic carbons at 149.74, 147.98, 147.50 ppm and two carboxyls at 171.23 and 169.89 ppm. The ¹³C NMR spectrum also contains a methoxyl at 48.50 ppm
The NMR data are consistent with the proposed structure of C-III, which also methanol contains in the sample. The molar ratio between C-III and methanol is approximately 1:0.94.

Infrared Spectroscopy

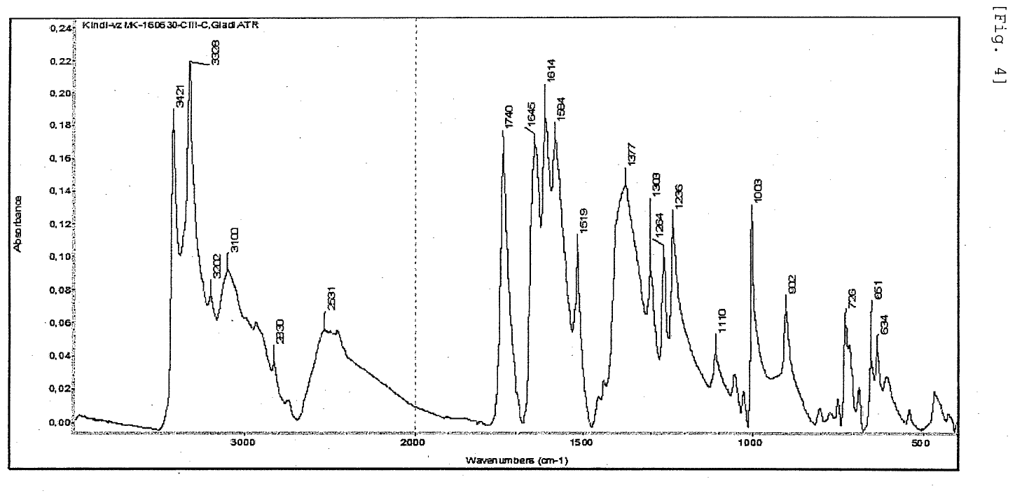

FTIR spectrometer Nicolet 6700 equipped with Continuum (T, R) microscope was used to measure infrared data of C-III. Data were captured ranging from 7500 to 350 cm⁻¹ and detected by DTGS/MCT-A with ATR supplement for reflection measurement. Bands and functional groups were assigned using UCT materials available online. The result is shown in FIG. 4.

Amide functional groups of the title compound are represented by peaks (all in cm⁻¹) 3421, 3202 and 1614. It is also typical for amide to form shoulder peaks in the vicinity of wavenumbers 1420-1400 and 1236-1150.
Amine FG shows two major signals at 3421 and 3328. This is further accompanied by 1614, 1003, 902 and 651. If amine is on an aromatic ring, a peak of this vibration is somewhere in the range of wavenumbers 1377-1236.
Carboxyl FG is represented by CO vibrations and OH vibrations. The former being present primarily at 1740 and, to lesser extent, at 2830. Hydroxyl should be presented at 2531. All applies to monomer COOH, which is being confirmed at 1377. Long shoulder 1236-1003 is typical for carboxyls as well.
Having full substitution on the aromatic ring present, there are no peaks through 2000-1800. Multiple bands are presented at 1645, 1614, 1584 and 1519, which is typical, but with varying intensities. In the fingerprint area, below 1000, a clear peak 725 supports the aromatic ring.
Iodine atoms should be presented only in fingerprint area only if their signals weren't mutually eliminated by their symmetry on the aromatic ring.
Blank methanol would have a strong peak 1900-1800. Having its oxygen forming a hydrogen bond with the C-III molecule, this shifts the signal all the way to 3100. This is typical for a chelating —OH bond. The bulk formed underneath (3500-2800) should confirm the methanol presence as well, forming a shoulder from 3328 to 3202. Minor peaks should be accounted for in 1110-1003.

### Example 4. Preparation of C-IV 3-amino-5-(aminocarbonyl)-2,4,6-triiodobenzoyl Chloride

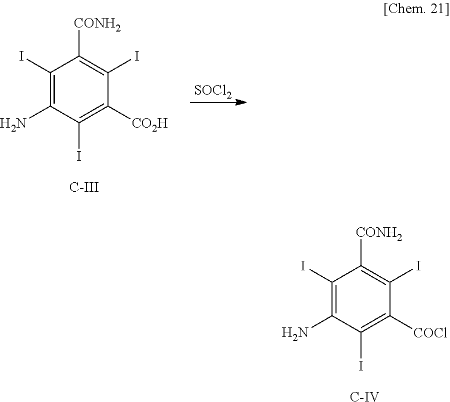

A 2500 mL three neck round-bottomed flask equipped with a magnetic stirrer was charged with the pure dewatered intermediate 3-amino-5-(aminocarbonyl)-2,4,6-triiodobenzoic acid, C-III (lot number: AK-CIV-223-250516, 338 grams, 0.60589 mol) and ethyl acetate (1813 mL), thionyl chloride (104 mL, 1.442 mol) and dimethylformamide (1.8 mL). The mixture was refluxed for 6 hours and then cooled to 40-45° C. Solvents were removed by vacuum distillation (1189 mL). Mixture was cooled to 25-30° C. and tetrahydrofuran (1553 mL) was added to the mixture. Mixture was extracted by a saturated solution of sodium chloride with sodium hydrogen carbonate (163 grams of sodium chloride, 91.4 grams sodium bicarbonate and 813 mL of demineralized water).

The second and third extraction of organic phase was made by a saturated solution of sodium chloride (269 grams and 938 mL demineralized water). After extraction, the solvent is distilled off from mixture (1520 mL). After adding of ethyl acetate (876 mL) the solvent was distilled off (770 mL) and mixture was cooled to 0-5° C. The precipitated solids were collected with a Nutsch filter funnel, followed by a wash using ethyl acetate (90 mL). The wet cake was transferred to glass drying trays and the solid was dried in a vacuum oven set to 55-60° C. under filtered nitrogen atmosphere to afford the intermediate C-IV as a white yellow powder of 3-amino-5-(aminocarbonyl)-2,4,6-triiodobenzoyl chloride (399.2 grams, lot number AK-CIV-223-250516). Chromatographic purity is 97.98% area. Yield is 85.87%. In a similar manner to the above procedure, some working examples were prepared, the results of which are shown in Table 4.

TABLE 4

| Purity of staring | Number | | | purity |
|---|---|---|---|---|
| C-III (purity of | of batch | | | of HPLC |
| HPLC area: | C-IV: | Batch of C-IV: | Yield: | area: |
| 99.72% | 1. | AK-CIV-223-250516 | 79.47% | 97.98% |
| 99.67% | 2. | AK-CIV-234-200616 | 87.19% | 96.79% |
| 99.67% | 3. | AK-CIV-238-290616 | 83.78% | 97.92% |
| 99.60% | 4. | AK-CIV-242-180716 | 79.52% | 98.27% |
| 99.50% | 5. | AK-CIV-243-200716 | 79.20% | 97.53% |
| 99.60% | 6. | AK-CIV-244-250716 | 79.16% | 98.09% |
| 99.73% | 7. | AK-CIV-245-270716 | 85.49% | 97.56% |

### Example 5. Purification of C-IV (1)

One gram of 3-amino-5-(aminocarbonyl)-2,4,6-triiodobenzoyl chloride, C-IV (batch: AK-CIV-238-290616 with impurity C-III 0.38%) is dissolved in tetrahydrofuran (10 mL) and stirred with anion resins (Purolite A-400, 2 grams) for 0.5 hour at 20-30° C. The mixture with resin was filtered and washed with tetrahydrofuran (10 mL). Filtered solution was concentrated on rotary evaporator. Chromatographic purity is 98.82% area. Yield is 0.9 grams of 3-amino-5-(aminocarbonyl)-2,4,6-triiodobenzoyl chloride, C-IV without impurity C-III. See Table 5 for results.

TABLE 5

| | | | Mixture | |
|---|---|---|---|---|
| | | | after deionization | |
| Mixture before deionization: | | | (% HPLC area): | |
| Used batch | | Impurity C-III | | Impurity: |
| of C-IV: | Purity: | (HPLC area): | Purity: | C-III |
| AK-CIV-238- | 97.92% | 0.38% | 98.82% | 0.00% |
| 290616 | | | | |

### Example 6. Purification of C-IV (2)

A mixture of 3-amino-5-(aminocarbonyl)-2,4,6-triiodobenzoyl chloride, C-IV (4 grams) and solvent (ration between C-VI and solvent is described in Table 6) was heated (50-80° C.) to the dissolution. Solution of C-IV was eventually concentrated under reduced pressure (400-220 mbar). Antisolvent was added during 20-30 minutes. Crystallized mixture was cooled (45-120 minutes) to 20-25° C. Crystals of 3-amino-5-(aminocarbonyl)-2,4,6-triiodobenzoyl chloride (C-IV) were filtered off, washed with solvent and dried at temperature of 50-60° C. Experiment results are summarized in Table 6.

TABLE 6

| | | Used solvent, | | | | |
|---|---|---|---|---|---|---|
| | | antisolvent | Used vacuum | | | |
| | | ratio | distilation? | | Purity | |
| | Used batch | C-III:solvent:antisolvent | Distill off | | (% of HPLC | |
| Entry | of C-IV: | (g.mL) | (mL) | Temp. | area) | Yield |
| 1. | AK-CIV-238- | C-III:THF:MTHF | No | 80° C. | 99.26% | 67.5% |
| | 290616 | 4:20:20 | | | | |
| 2. | AK-CIV-238- | C-III:THF:MTHF:heptane | No | 80° C. | 99.20% | 78.7% |
| | 290616 | 4:15:5:10 | | | | |
| 3. | AK-CIV-244- | C-III:THF:MTHF:heptane | No | 80° C. | 98.58% | 85.2% |
| | 250716 | 4:15:5:20 | | | | |
| 4. | AK-CIV-234- | C-III:THF:MTHF:heptane | No | 80° C. | 97.99% | 84.3% |
| | 200616 | 4:15:5:20 | | | | |
| 5. | AK-CIV-234- | C-III:Dioxan:heptane | No | 100° C. | 96.56% | 89.0% |
| | 200616 | 4:14:20 | | | | |
| 6. | AK-CIV-234- | C-III:THF:hexane | No | 80° C. | 96.74% | 87.1% |
| | 200616 | 4:20:20 | | | | |
| 7. | AK-CIV-234- | C-III:THF:MTHF:heptane | Yes | 66° C. | 96.72% | 80.2% |
| | 200616 | 10:38:12:50 | 3 mL | | | |
| 8. | AK-CIV-238- | C-III:THF:toluene | Yes | 50° C. | 99.12% | 74.8% |
| | 290616 | 10:38:12:50 | 18 mL | | | |
| 9. | AK-CIV-238- | C-III:THF | Yes | 80° C. | 99.47% | 38.0% |
| | 290616 | 10:50 | 30 mL | | | |
| 10. | AK-CIV-238- | C-III:THF:toluene | Yes | 80° C. | 99.28% | 77.8% |
| | 290616 | 10:50:10 | 23 mL | | | |
| 11. | AK-CIV-234- | C-III:THF:toluene | Yes | 80° C. | 97.41% | 79.4% |
| | 200616 | 10:50:10 | 28 mL | | | |

### Example 7. Preparation of C-V (1)

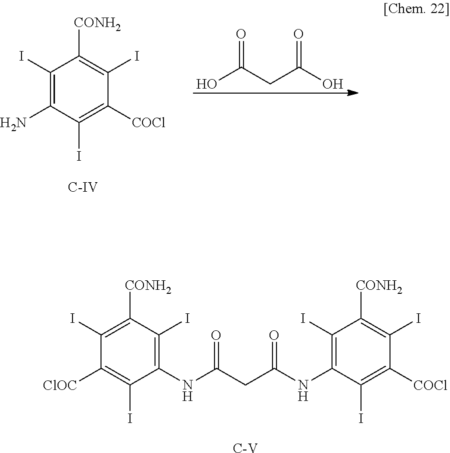

A 2500 mL three neck round-bottomed flask equipped with a mechanic stirrer was charged with the intermediate 3-amino-5-(aminocarbonyl)-2,4,6-triiodobenzoyl chloride, C-IV (lot number: AK-CIV-244-250716, 200 grams, 0.347 mol) and freshly distilled tetrahydrofuran (1197 mL), malonic acid (18.36 grams, 0.176 mol). The stirred mixture was heated to temperature of 45-50° C. and solution of phosphorus trichloride (26.3 grams, 0.192 mol) and freshly distilled tetrahydrofuran (200 mL) was continuously added to the reaction mixture during 3 hours. The reaction mixture was heated for 17 hours at 45-50° C. The mixture was cooled to 5-10° C. The precipitated solids were collected with a Nutsch filter funnel, followed by washing using freshly distilled tetrahydrofuran (4×160 mL). The wet cake was transferred to glass drying trays and the solid was dried in a vacuum oven set to 45-50° C. under filtered nitrogen atmosphere to afford 195.74 g of 3,3′-[(1,3-dioxo-1,3-propanediyl)diimino]bis[5-(aminocarbonyl)-2,4,6-triiodobenzoyl chloride] (batch number: AK-6-031016) which had a chromatographic purity of 98.99 area %. Yield is 92.4%. In a similar manner to the above procedure, some working examples were prepared, the results of which are shown in Table 7.

TABLE 7

| | | Purity of | | Purity of | |
|---|---|---|---|---|---|
| | | C-IV | | C-V | |
| | Used batch | (% HPLC | Coupling | (% HPLC | |
| Entry | of C-IV | area) | agent | area) | Yield |
| AK-226- | AK-CIV-223- | 97.98% | PCl₃ | 96.69% | 92.0% |
| 300516 | 250516 | | | | |
| AK-246- | AK-CIV-243- | 97.53% | PCl₃ | 99.24% | 90.9% |
| 220816 | 200716 | | | | |
| AK-247- | AK-CIV-223- | 97.98% | PCl₃ | 99.08% | 93.6% |
| 250816 | 250516 | | | | |
| | AK-CIV-243- | 97.53% | | | |
| | 200716 | | | | |
| AK-6- | AK-CIV-244- | 98.09% | PCl₃ | 98.99% | 92.4% |
| 031016 | 250716 | | | | |
| AK-7- | AK-CIV-234- | 96.79% | PCl₃ | 96.29% | 91.8% |
| 061016 | 200616 | | | | |
| AK-8- | AK-CIV-245- | 97.56% | PCl₃ | 98.53% | 91.5% |
| 101016 | 270716 | | | | |
| AK-9- | AK-CIV-238- | 97.92% | PCl₃ | 98.58% | 91.5% |
| 131016 | 290616 | | | | |
| | AK-CIV-245- | 97.56% | | | |
| | 270716 | | | | |
| AK-10- | AK-CIV-242- | 98.27% | T3P* | 97.16% | 92.1% |
| 181016 | 180716 | | | | |

*T3P = 1-propanephosphonic acid cyclic anhydride

### Example 8. Preparation of C-V (2)

A 2500 mL three neck round-bottomed flask equipped with a mechanic stirrer was charged with the intermediate 3-amino-5-(aminocarbonyl)-2,4,6-triiodobenzoyl chloride (C-IV) (lot number: AK-CIV-242-180716, 200 grams, 0.347 mol), freshly distilled tetrahydrofuran (1150 mL) and malonic acid (18.4 grams, 0.176 mol). The stirred mixture was heated to 48-50° C. and 50% wt. solution of 1-propanephosphonic acid cyclic anhydride in ethyl acetate (287.3 grams, 0.455 mol) was continuously added to the reaction mixture during 1 hours. The reaction mixture was heated for 17 hours at 50° C. The mixture was cooled to 25° C. The precipitated solids were collected on filter funnel, followed by a washing using freshly distilled tetrahydrofuran (2×200 mL). The wet cake was transferred to glass drying trays and the solid (376 grams) was dried in a vacuum oven at 48-50° C. under nitrogen atmosphere. 194.3 g (yield is 92.1%) of 3,3′-[(1,3-dioxo-1,3-propanediyl)diimino]bis[5-(aminocarbonyl)-2,4,6-triiodobenzoyl chloride] (C-V) (batch number: AK-10-181016) was obtained with a chromatographic purity 97.16 area %.

### Example 9. Preparation of C-VI

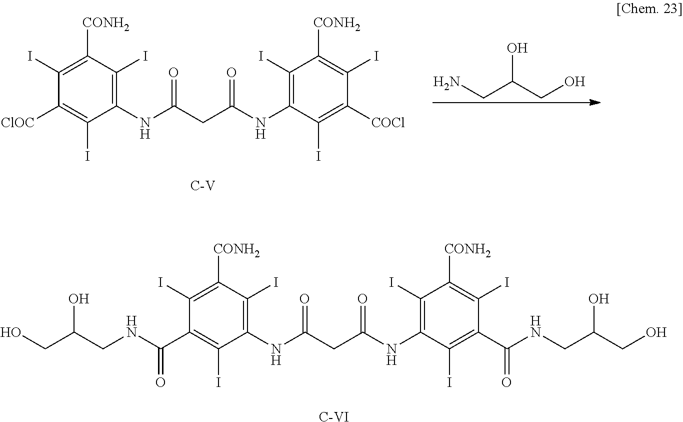

A 2500 mL three neck round-bottomed flasks equipped with a magnetic stirrer was charged with the intermediate C-V (3,3′-[(1,3-dioxo-1,3-propanediyl)diimino]bis[5-(aminocarbonyl)-2,4,6-triiodobenzoyl chloride]), lot number: AK-9-131016, 387.1 grams of wet compound (198.2 gram (of dried compound), 0.162 mol), dimethylformamide (396 mL) and triethylamine (39.59 grams). The mixture was cooled to 2-10° C. Then a solution 3-amino-propane-1,2-diol (35.35 grams, 0.388 mol) in dimethylformamide (77.7 mL) was added dropwise over ca. 90 minutes, maintaining the internal temperature at 2-8° C. The reaction mixture was stirred for 23 hours at 5-25° C. The reaction mixture was filtered with diatomaceous earth (2. grams) and washed by dimethylformamide (50 mL), and the filtered solution was stirred with anion resins (Purolite A-400, 153 grams) and methanol (100 mL) and water (100 mL) for half hour at 20-30° C. The mixture with resin was filtered and washed with water (100 mL). The filtered solution was again stirred with anion resins (Purolite A-400, 68 grams) for one hour at 20-30° C. Then the mixture with resin was filtered and washed with water (50 mL). The filtrate was precipitated.

A 6000 mL three neck round-bottomed flask equipped with a magnetic stirrer was charged with solution of C-VI. The solution was stirred and heated to 60-65° C. Then a isopropanol (1350 mL) and solution of acetic acid in isopropanol (acetic acid 166 mL and isopropanol 249 ml, pH check—it has to be 5-7) was added dropwise over ca. 90 minutes, maintaining the internal temperature at 60-65° C. The mixture was slowly cooled to temperature of 10° C. and stirred for another 2 hours. The mixture was cooled to 5-10° C. and filtered. (Time of cooling was 1 hour). The resulting solids were collected by vacuum filtration through a filter funnel and the product cake was resuspended in acetone (1200 mL) under stirring over half hour at 20-25° C. Solids were collected by vacuum filtration through a filter funnel and the product cake was washed with acetone (150 mL). Then wet cake was transferred to glass drying trays and the solid was dried in a vacuum oven set to 55-60° C. under filtered nitrogen atmosphere to afford the 5,5′-[(1,3-dioxo-1,3-propanediyl)diimino]bis[N-(2,3-dihydroxypropyl)-2,4,6-triiodo-1, 3-benzenedicarboxamide], C-VI as a white off powder (173.1 grams, yield 83%, purity 98.0%, batch number JM-071216).

### Example 10. Preparation of C-VI Diacetonide (1)

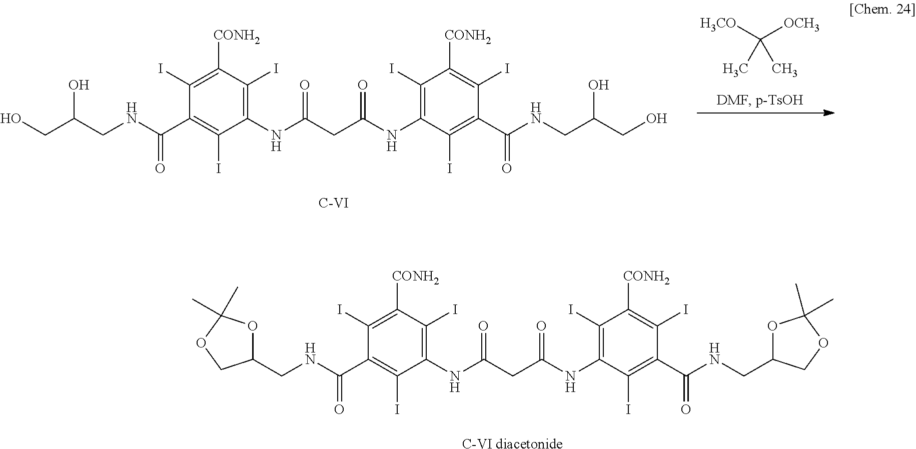

Under argon (just exclusion of air moisture would be enough), in a 2 L round bottom flask with 1 inch egg-shaped stir bar was suspended C-VI (5,5′-[(1,3-dioxo-1,3-propanediyl)diimino]bis[N-(2,3-dihydroxypropyl)-2,4,6-triiodo-1,3-benzenedicarboxamide]) (40 grams, 0.0301 mol) in N,N-dimethylformamide, (200 ml). To the stirred suspension, 2,2-dimethoxypropane (40 mL, 0.325 mol, 10.8 eq.) and pTsOH.H₂O (3 grams, 0.0166 mol, 0.55 eq.) were added. After heating for 15-20 min to 30-40° C. until a cloudy solution was obtained, the mixture was stirred at room temperature for 16 hrs. To the resulting light yellow, slightly cloudy solution was added TEA (3 ml, 0.0215 mol, 0.7 eq., pH check—it has to be over 7).

Crystallization of C-VI Diacetonide from the Reaction Mixture:

Water (150 ml) was slowly added (ca 4 min, temperature spontaneously increased to ca 30° C.) until the first cloudiness appeared. Additional water (130 ml) was dropwise added over the period of 1 hr. The crystallizing mixture was stirred for 1 hr. Additional water (320 ml) was dropwise added over the period of 15 min. The crystallizing mixture was stirred for 3 hrs. Additional water (600 ml, to the total of 1200 mL) was dropwise added over the period of 2 hours. The crystallizing mixture was stirred for 3 hrs.

### Isolation:

The solid product was filtered off and washed gradually with water (300 mL) and 2-propanol (40 mL). After drying on open air for 2 days, it was obtained 39.0 grams of C-VI diacetonide as a colorless, fine crystalline product. Yield was 92% and chromatographic purity of 98.6 area %.

### Example 11. Preparation of C-VI Diacetonide (2)

A 500 mL three neck round-bottomed flasks equipped with a magnetic stirrer was charged with the intermediate 3,3′-[(1,3-dioxo-1,3-propanediyl)diimino]bis[5-(aminocarbonyl)-2,4,6-triiodobenzoyl chloride], C-V (36.71 grams of dried compound, 30.07 mmol), dimethylformamide (110 mL) and triethylamine (6.36 grams, 62.85 mmol). The mixture was cooled to 2-10° C. Then a solution 3-amino-propane-1,2-diol (5.67 grams, 62.25 mmol) in dimethylformamide (90 mL) was added dropwise over ca. 90 minutes, maintaining the internal temperature at 2-8° C. The reaction mixture was stirred for 16 hours at 5-25° C. The reaction mixture was filtered and filter cake of triethylamine hydrochloride was washed by dimethylformamide (50 mL). The filtrate was charged in 500 mL three neck round-bottomed flasks equipped with a magnetic stirrer and acidified by solution of hydrogen chloride in diethyl ether (26 wt %) to pH=2.0 at 200-25° C. Then, 2,2-dimethoxypropane (40 mL, 325 mmol) and p-toluenesulfonic acid monohydrate (3 grams, 16.6 mmol) were added. After heating for 15-20 min to 30-40° C. until a cloudy solution was obtained, the mixture was stirred at room temperature for 16 hrs. To the resulting light yellow, slightly cloudy solution was added TEA (3 ml, 0.0215 mol, pH check—it has to be over 7).

Crystallization of C-VI Diacetonide from the Reaction Mixture:

Water (150 ml) was slowly added (ca 4 min, temperature spontaneously increased to ca 30° C.) until the first cloudiness appeared. Additional water (130 ml) was dropwise added over the period of 1 hr. The crystallizing mixture was stirred for 1 hr. Additional water (320 ml) was dropwise added over the period of 15 min. The crystallizing mixture was stirred for 3 hrs. Additional water (600 ml, to the total of 1200 ml) was dropwise added over the period of 2 hours. The crystallizing mixture was stirred for 3 hrs.

Isolation:

The solid product was filtered off and washed gradually with water (300 mL) and 2-propanol (40 mL). After drying on open air, it was obtained 33.0 grams of C-VI diacetonide as a colorless, fine crystalline product. Yield was 78% and chromatographic purity of 89.71 area %.

### Example 12. Preparation of Iosimenol from C-VI (Using 2-Methoxyethanol as a Reaction Solvent)

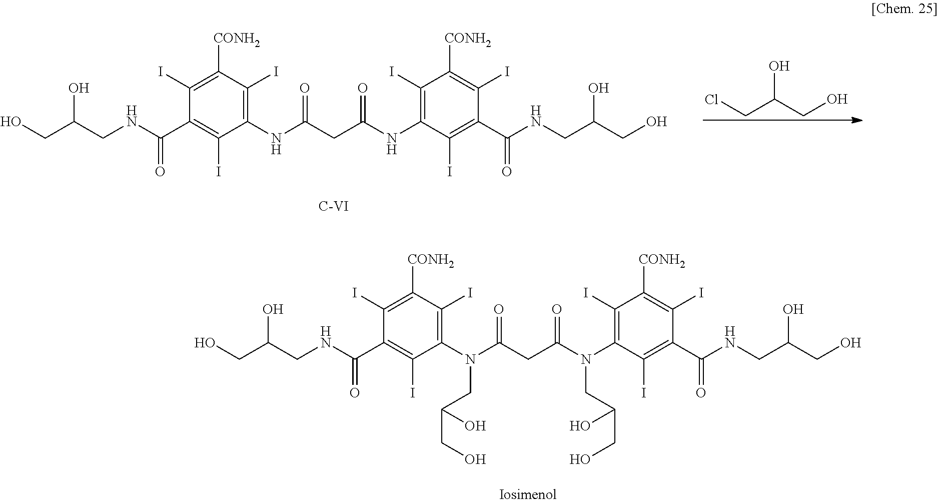 [SMILES: NC(=O)c1c(I)c(NC(=O)CC(=O)Nc2c(I)c(C(N)=O)c(I)c(C(=O)NCC(O)CO)c2I)c(I)c(C(=O)NCC(O)CO)c1I ; OCC(O)CCl ; "C-VI" ; NC(=O)c1c(I)c(C(=O)NCC(O)CO)c(I)c(N(CC(O)CO)C(=O)CC(=O)N(CC(O)CO)c2c(I)c(C(N)=O)c(I)c(C(=O)NCC(O)CO)c2I)c1I]

A 100 mL three neck round-bottomed flask equipped with a magnetic stirrer was charged with the intermediate C-VI, 5,5′-[(1,3-dioxo-1,3-propanediyl)diimino]bis[N-(2,3-dihydroxypropyl)-2,4,6-triiodo-1, 3-benzenedicarboxamide] (lot number: AK-11-201216A, 10.0 grams (0.00752 mol)), 2-methoxyethanol (30 mL), lithium hydroxide (0.719 grams, 0.0307 mol), glycerin (2.5 grams) and anhydrous calcium chloride (3.18 grams, 0.0284 mol). The mixture was heated to 40-45° C. Then a 50% wt. solution of 3-chloro-propane-1,2-diol in 2-methoxyethanol (3.15 grams of 3-chloro-propane-1,2-diol, 0.0286 mol) was added gradually during 7 hours, maintaining the internal temperature at 36-38° C. The reaction mixture was heated for 21 hours at 36-38° C. After this time, the reaction was considered complete; the reaction mixture was precipitated by ethanol (60 mL) at 55-60° C. Cooled suspension (at 20-25° C.) was filtered and washed with methanol (50 mL). The crude product with salts contained 10.2 grams of iosimenol (92% theory). Then the solid was transferred to glass drying tray and the solid was dried in a vacuum oven set to 55-60° C. under filtered nitrogen atmosphere to afford the (5,5′-[(1,3-dioxo-1,3-propanediyl)bis[(2,3-dihydroxypropyl)imino]]bis[N-(2,3-dihydroxypropyl)-2,4,6-triiodo-1,3-benzenedicarboxamide]), crude iosimenol as a white off powder (15.85 grams, 92% of theory), HPLC purity: 94.11 area %. Overalkyls: impurity IMP1+IMP2 2.51 area %, impurity IMP3 0.28 area % and impurity IMP4 0.47 area % (supported by results of batch number: JM-291116A).

### Example 13. Preparation of Iosimenol from C-VI (Using 2-Methoxyethanol as a Reaction Solvent, Bigger Scale)

A 1 L three neck round-bottomed flask equipped with a mechanical stirrer was charged with the intermediate C-VI, 5,5′-[(1,3-dioxo-1,3-propanediyl)diimino]bis[N-(2,3-dihydroxypropyl)-2,4,6-triiodo-1, 3-benzenedicarboxamide] (lot number: AK-124-171017K, 100.0 grams (0.0752 mol)), 2-methoxyethanol (300 mL), lithium hydroxide monohydrate (12.62 grams, 0.3007 mol), glycerol anhydrous (25 grams) and anhydrous calcium chloride (31.8 grams, 0.2865 mol). The mixture was heated to 40-45° C. Then a 50% wt. solution 3-chloro-propane-1,2-diol in 2-methoxyethanol (31.6 grams of 3-chloro-propane-1,2-diol, 0.2865 mol) was added gradually during 4.5 hours, maintaining the internal temperature at 40-45° C. Reaction mixture was stirred 17 hours at 40-45° C. Then the second portion of a 50% wt. solution 3-chloro-propane-1,2-diol in 2-methoxyethanol (7.9 grams of 3-chloropropane-1,2-diol, 0.0715 mol) was added (gradually during 30 minutes). The reaction mixture was stirred for additional 5 hours at 40-45° C. and course of reaction was monitored by HPLC. Reaction mixture was precipitated by ethanol (600 mL) within 30 minutes at 40-45° C. Cooled suspension was filtered at 20-25° C. and filter cake was washed with ethanol (100 mL), reslurred with methanol (600 ml) and stirred 15 minutes at 20-25° C. and suspension filtered again. The obtained solid was washed with ethanol (300 mL), transferred into glass drying tray and dried in a vacuum at 55-60° C. under nitrogen atmosphere to afford the (5,5′-[(1,3-dioxo-1,3-propanediyl)bis[(2,3-dihydroxypropyl)imino]]bis[N-(2,3-dihydroxypropyl)-2,4,6-triiodo-1,3-benzenedicarboxamide]), crude iosimenol as a white off powder with salts (108 grams contained 99.99 grams of iosimenol, yield 90% of theory), HPLC purity: 92.95 area %. Overalkyls: impurity IMP1+IMP2 2.51 area %, impurity IMP3 0.27 area % and impurity IMP4 0.60 area % (supported by results of batch number: AK-126-301017)

### Example 14. Preparation of Iosimenol from C-VI (Using Dimethyl Sulfoxide as a Reaction Solvent)

A 50 mL three neck round-bottomed flask equipped with a magnetic stirrer was charged with the intermediate C-VI, 5,5′-[(1,3-dioxo-1,3-propanediyl)diimino]bis[N-(2,3-dihydroxypropyl)-2,4,6-triiodo-1, 3-benzenedicarboxamide] (lot number: JM-060612, 5.0 grams (0.00375 mol)), dimethyl sulfoxide (20 mL), lithium hydroxide monohydrate (0.631 grams, 0.01504 mol), glycerol anhydrous (1.75 grams) and anhydrous calcium chloride (1.69 grams, 0.01504 mol). The mixture was heated to 40-45° C. Then a 3-chloro-propane-1,2-diol in 2-methoxyethanol (1.66 grams, 0.01504 mol) was added, maintaining the reaction temperature at 33-37° C. Reaction mixture was stirred 60 hours at 33-37° C. Then 3-chloro-propane-1,2-diol (2.91 grams, 0.02632 mol) was added. The reaction mixture was stirred for an additional 178 hours at 33-37° C. The reaction mixture was monitored by HPLC. After this time, the reaction was considered complete and precipitated by ethanol (120 mL) during 30 minutes at 33-37° C. Cooled suspension was filtered at 20-25° C. and the obtained solid was washed with ethanol (20 mL), transferred to glass drying tray and dried in a vacuum at 55-60° C. under nitrogen atmosphere to afford the (5,5′-[(1,3-dioxo-1,3-propanediyl)bis[(2,3-dihydroxypropyl)imino]]bis[N-(2,3-dihydroxypropyl)-2,4,6-triiodo-1,3-benzenedicarboxamide]), crude iosimenol as a yellowish powder contained 2.28 grams of iosimenol with HPLC purity: 90.32 area %. Overalkyls: impurity IMP1+IMP2 2.34 area %, impurity IMP3 0.11 area % and impurity IMP4 1.25 area % (supported by results of batch number: JM-060317G). Yield 41% of theory.

### Example 15. Preparation of Iosimenol from C-VI (Using N,N-Dimethylformamide as a Reaction Solvent)

A 50 mL three neck round-bottomed flask equipped with a magnetic stirrer was charged with the intermediate C-VI, 5,5′-[(1,3-dioxo-1,3-propanediyl)diimino]bis[N-(2,3-dihydroxypropyl)-2,4,6-triiodo-1, 3-benzenedicarboxamide] (lot number: JM-060612, 5.0 grams (0.00375 mol)), N,N-dimethylformamide (20 mL), lithium hydroxide monohydrate (0.631 grams, 0.01504 mol), glycerol anhydrous (1.75 grams) and anhydrous calcium chloride (1.69 grams, 0.01504 mol). The mixture was heated to 40-45° C. Then 3-chloro-propane-1,2-diol (1.66 grams, 0.01504 mol) was added, maintaining temperature at 33-37° C. Reaction mixture was stirred for 67 hours at 33-37° C. Then additional 3-chloro-propane-1,2-diol (0.83 grams, 0.00752 mol) was added. The reaction mixture was stirred for an additional 27 hours at 33-37° C. The course of reaction was monitored by HPLC. Then the reaction mixture was precipitated by ethanol (120 mL) during 30 minutes at 33-37° C. Cooled suspension was filtered at 20-25° C. and the obtained solid was washed with ethanol (20 mL), transferred to glass drying tray and dried in a vacuum at 55-60° C. under nitrogen atmosphere to afford the (5,5′-[(1,3-dioxo-1,3-propanediyl)bis[(2,3-dihydroxypropyl)imino]]bis[N-(2,3-dihydroxypropyl)-2,4,6-triiodo-1,3-benzenedicarboxamide]), crude iosimenol as a yellowish powder contained 3.30 grams of iosimenol with HPLC purity: 90.60 area %. Overalkyls: impurity IMP1+IMP2 1.41 area %, impurity IMP3 0.10 area % and impurity IMP4 0.86 area % (supported by results of batch number: JM-060317H). Yield 59.5% of theory.

### Example 16. Preparation of Iosimenol from C-VI (Using N-Methyl-2-Pyrrolidone as a Reaction Solvent)

A 50 mL three neck round-bottomed flask equipped with a magnetic stirrer was charged with the intermediate C-VI, 5,5′-[(1,3-dioxo-1,3-propanediyl)diimino]bis[N-(2,3-dihydroxypropyl)-2,4,6-triiodo-1, 3-benzenedicarboxamide] (lot number: AK-51-240317K, 5.0 grams (0.00375 mol)), N-methyl-2-pyrrolidone (15 mL), lithium hydroxide monohydrate (0.631 grams, 0.01504 mol), glycerol anhydrous (1.75 grams) and anhydrous calcium chloride (1.69 grams, 0.01504 mol). The mixture was heated to 40-45° C. Then 3-chloro-propane-1,2-diol (2.50 grams, 0.0226 mol) was added, maintaining the temperature at 33-37° C. Reaction mixture was stirred for 60 hours at 33-37° C. Then additional 3-chloro-propane-1,2-diol (0.83 grams, 0.00752 mol) was charged into the reaction. Reaction mixture was stirred for an additional 36 hours at 33-37° C. The course of reaction was monitored by HPLC. The reaction mixture was precipitated by ethanol (120 mL) during 30 minutes at 33-37° C. The obtained suspension was filtered at 20-25° C. and the obtained solid was washed with ethanol (20 mL), transferred to glass drying tray and dried in a vacuum at 55-60° C. under nitrogen atmosphere to afford the (5,5′-[(1,3-dioxo-1,3-propanediyl)bis[(2,3-dihydroxypropyl)imino]]bis[N-(2,3-dihydroxypropyl)-2,4,6-triiodo-1,3-benzenedicarboxamide]), crude iosimenol as a yellowish powder contained 4.17 grams of iosimenol with HPLC purity: 92.34 area %. Overalkyls: impurity IMP1+IMP2 2.21 area %, impurity IMP3 0.20 area % and impurity IMP4 1.04 area % (supported by results of batch number: JM-310317AKR). Yield 75% of theory.

### Example 17. Preparation of Iosimenol from C-VI (Using Ethylene Glycol as a Reaction Solvent)

A 50 mL three neck round-bottomed flask equipped with a magnetic stirrer was charged with the intermediate C-VI, 5,5′-[(1,3-dioxo-1,3-propanediyl)diimino]bis[N-(2,3-dihydroxypropyl)-2,4,6-triiodo-1, 3-benzenedicarboxamide] (lot number: JM-060612, 5.0 grams (0.00375 mol)), ethylene glycol (20 mL), lithium hydroxide monohydrate (0.631 grams, 0.01504 mol), glycerol anhydrous (1.75 grams) and anhydrous calcium chloride (1.69 grams, 0.01504 mol). The mixture was heated to 40-45° C. Then 3-chloro-propane-1,2-diol (1.66 grams, 0.01504 mol) was added, maintaining temperature at 33-37° C. Reaction mixture was stirred for 60 hours at 33-37° C. Then additional 3-chloro-propane-1,2-diol (0.83 grams, 0.00752 mol) was charged into reaction mixture and stirred for additional 27 hours at 33-37° C. The course of reaction was monitored by HPLC. Reaction mixture was precipitated by ethanol (120 mL) during 30 minutes at 33-37° C. The obtained suspension was filtered at 20-25° C. and the obtained solid was washed with ethanol (20 mL), transferred to glass drying tray and dried in a vacuum at 55-60° C. under nitrogen atmosphere to afford the (5,5′-[(1,3-dioxo-1,3-propanediyl)bis[(2,3-dihydroxypropyl)imino]]bis[N-(2,3-dihydroxypropyl)-2,4,6-triiodo-1,3-benzenedicarboxamide]), crude iosimenol as a yellowish powder contained 2.76 grams of iosimenol with HPLC purity: 85.52 area %. Overalkyls: impurity IMP1+IMP2 3.47 area %, impurity IMP3 0.35 area % and impurity IMP4 3.04 area % (supported by results of batch number: JM-060317F). Yield 49.72% of theory.

### Example 18. Preparation of Iosimenol from C-VI (Using Propylene Glycol as a Reaction Solvent)

A 50 mL three neck round-bottomed flask equipped with a magnetic stirrer was charged with the intermediate C-VI, 5,5′-[(1,3-dioxo-1,3-propanediyl)diimino]bis[N-(2,3-dihydroxypropyl)-2,4,6-triiodo-1, 3-benzenedicarboxamide] (lot number: JM-060612, 5.0 grams (0.00375 mol)), propylene glycol (20 mL), lithium hydroxide monohydrate (0.631 grams, 0.01504 mol), glycerol anhydrous (1.75 grams) and anhydrous calcium chloride (1.69 grams, 0.01504 mol). The mixture was heated to 40-45° C. Then 3-chloro-propane-1,2-diol (1.66 grams, 0.01504 mol) was added, maintaining temperature at 33-37° C. Reaction mixture was stirred for 60 hours at 33-37° C. Then additional 3-chloro-propane-1,2-diol (0.83 grams, 0.00752 mol) was charged into reaction mixture and stirred for additional 27 hours at 33-37° C. The course of reaction was monitored by HPLC. Reaction mixture was precipitated by ethanol (120 mL) during 30 minutes at 33-37° C. The obtained suspension was filtered at 20-25° C. and obtained solid was washed with ethanol (20 mL), transferred to glass drying tray and dried in a vacuum at 55-60° C. under nitrogen atmosphere to afford the (5,5′-[(1,3-dioxo-1,3-propanediyl)bis[(2,3-dihydroxypropyl)imino]]bis[N-(2,3-dihydroxypropyl)-2,4,6-triiodo-1,3-benzenedicarboxamide]), crude iosimenol as a yellowish powder contained 3.42 grams of iosimenol with HPLC purity: 88.18 area %. Overalkyls: impurity IMP1+IMP2 1.95 area %, impurity IMP3 0.24 area % and impurity IMP4 0.96 area % (supported by results of batch number: JM-060317E). Yield 61.6% of theory.

### Example 19. Preparation of Iosimenol from C-VI Diacetonide

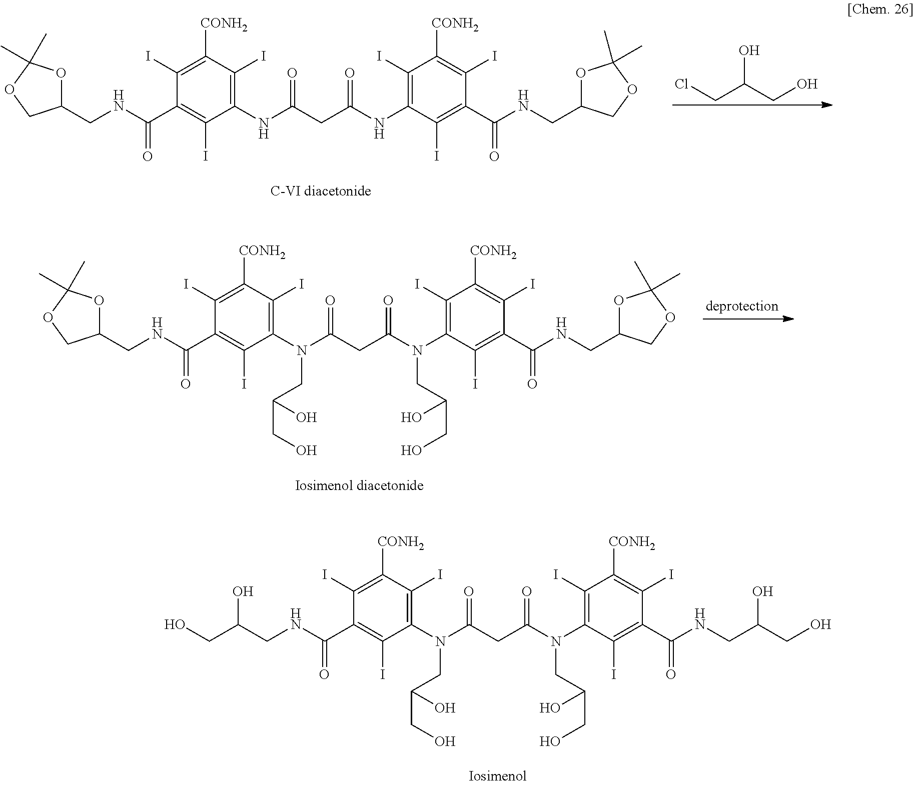

A 100 mL three neck round-bottomed flask equipped with a magnetic stirrer was charged with C-VI diacetonide (5,5′-(malonylbis(azanediyl))bis(N-((2,2-dimethyl-1,3-dioxolan-4-yl)methyl)-2,4,6-trii odoisophthalamide), lot number MM-21112016-224, 10.0 grams (0.00709 mol), methanol (24 mL), lithium hydroxide monohydrate (1.22 grams, 0.0291 mol), glycerin (1.5 grams) and anhydrous calcium chloride (3.13 grams, 0.0282 mol) and 3-chloro-propane-1,2-diol (3.13 grams, 0.0283 mol). The mixture was heated to 40-45° C. for 24 hours. After this time, sample (0.2 mL) of the reaction mixture was for 1 min heated to reflux with 5% hydrochloric acid (0.2 ml) neutralized with 5% ammonia and analyzed by HPLC: Iosimenol 91.93 area %, C-VI: 0.21 area %, impurity Monoalkyls 0.27 area %, impurities IMP1+IMP2 1.93 area %. The reaction mixture was evaporated at 35° C. to a sticky-foamy semisolid to which was added 2-propanol (26 ml). Slurry, obtained after 6 hours of stirring at room temperature, was cooled in refrigerator for 16 hours, filtered, washed with 2-propanol (10 mL) and dried. 10.46 grams of light yellow solid product with salts (yield 94%). HPLC analysis of iosimenol (after deprotection of iosimenol-diacetonide): Iosimenol ((5,5′-[(1,3-dioxo-1,3-propanediyl)bis[(2,3-dihydroxypropyl)imino]]bis[N-(2,3-dihydroxypropyl)-2,4,6-triiodo-1,3-benzenedicarboxamide])), 94.0 area %, C-VI 0.12 area %, impurity Monoalkyls 0.08 area %, impurities IMP1+IMP2 1.62 area % (supported by results of batch number: MM-21122016-278).

### Example 20. Purification of Iosimenol (1)

Deionized iosimenol (40 grams, purity 97.79% HPLC area) was suspended in solvent mixture containing 2-methoxyethanol (70 mL) and water (10 mL) and tromethamol (0.05 grams) in a 250 mL three-necked flask equipped with a condenser. The suspension was stirred and heated up to 90° C. until clear solution is obtained, then heated to reflux and seeds of iosimenol crystals (0.25 grams) were added. The mixture was maintained under reflux. The first crystals appeared after 12-16 hours. The antisolvent (1-methoxy-2-propanol, 30 mL) was added in two portion (one portion 15 mL of 1-methoxy-2-propanol—addition time approx. 90 min each). The first portion was added at the second day of crystallization. The course of crystallization was checked by monitoring of remaining dissolved iosimenol in sample of liquid phase and when remaining iosimenol stayed unchanged in two consecutive testing, the suspension was filtered at 80-90° C. The obtained solid material was washed with ethanol (100 mL) at 60-70° C. The HPLC purity of the iosimenol crystals were 98.5% and the crystallization yields were 85%. Total time of the crystallizations were 68 hours. In a similar manner to the above procedure, some working examples were prepared, the results of which are shown in Table 8.

TABLE 8

| | Experiment: | | | | | | | | | |
|---|---|---|---|---|---|---|---|---|---|---|
| | JM- | | JM- | | JM- | | JM- | | JM- | |
| | 091115/3 | | 241115/1 | | 241115/2 | | 241115/3 | | 241115/4 | |
| Starting | 40 g, | | 40 g, | | 40 g, | | 40 g, | | 40 g, | |
| Iosimenol purity: | 97.79% | | 97.79% | | 97.79% | | 97.79% | | 97.79% | |
| Batch of starting | AK-16- | | AK-16- | | AK-16- | | RK-16- | | AK-16- | |
| material | 130212 | | 130212 | | 130212 | | 130212 | | 130212 | |
| 2-Methoxyethanol | 70 | mL | 50.0 | mL | 50.0 | mL | 50.0 | mL | 50.0 | mL |
| Methoxy-2- | 30.0 | mL | 0.0 | mL | 0.0 | mL | 0.0 | mL | 0.0 | mL |
| propanol | | | | | | | | | | |
| Water | 10.0 | mL | 10.0 | mL | 10.0 | mL | 10.0 | mL | 10.0 | mL |
| Trometamol | 0.07 | g | 0.2 | g | 0.15 | g | 0.10 | g | 0.05 | g |
| Addition of | 0.0 | mL | 20.0 | mL | 20.0 | mL | 20.0 | mL | 20.0 | mL |
| methoxyethanol** | | | | | | | | | | |
| Gradient addition | 0.0 | mL | 60.0 | mL | 60.0 | mL | 60.0 | mL | 60.0 | mL |
| of 2- | | | | | | | | | | |
| ethoxyethanol** | | | | | | | | | | |
| Gradient add. of | 45.0 | mL | 0.0 | mL | 0.0 | mL | 0.0 | mL | 0.0 | mL |
| methoxy-2- | | | | | | | | | | |
| propanol** | | | | | | | | | | |
| Addition of water | 6.5 | mL | 0.0 | mL | 0.0 | mL | 0.0 | mL | 0.0 | mL |
| Tested | 107-111° | C. | 89-107° | C. | 89-107° | C. | 89-107° | C. | 89-107° | C. |
| temperature | | | | | | | | | | |
| range: | | | | | | | | | | |
| Seeding: | 0.25 | g | 0.26 | g | 0.3 | g | 0.3 | g | 0.3 | g |
| Crystallization | yes | | yes | | yes | | yes | | yes | |
| observed: | | | | | | | | | | |
| Total time of | 77 | hours | 70 | hours | 70 | hours | 70 | hours | 70 | hours |
| crystallization | | | | | | | | | | |

| | | | | | |
|---|---|---|---|---|---|
| Purity of | 98.76% | 98.53% | 98.85% | 98.58% | 98.35% |
| obtained crystal | | | | | |
| (% Area of HPLC) | | | | | |
| Yield | 66.7% | 70.0% | 66.2% | 78.0% | 85.0% |

**gradient addition of solvents started after 24 hours of crystallization

### Example 21. Purification of Iosimenol (2)

Deionized iosimenol (40 grams, purity 97.67% HPLC area) was suspended in solvent mixture containing glycols (diethylene glycol or triethylene glycol, 70 mL) and water (5 mL) and tromethamol (0.25 grams) in a 250 mL three-necked flask equipped with a condenser. The suspension was stirred and heated up to 90° C. until clear solution is obtained, then heated to reflux and seeds of iosimenol crystals (0.25 grams) were added. The mixture was maintained under reflux. The first crystals appeared after 12-24 hours. The antisolvent (isopropanol, 75 mL) was added in six portion (one portion 12.5 mL of isopropanol—addition time approx. 30 min each). The first portion was added at the second day of crystallization. The course of crystallization was checked by monitoring of remaining dissolved iosimenol in sample of liquid phase and when remaining iosimenol stayed unchanged in two consecutive testing, the suspension was filtered at 80-90° C. The obtained solid material was washed with ethanol (100 mL) at 60-70° C. The HPLC purity of the Iosimenol crystals were 94.8 and 98.5% and the crystallization yields were 48.0 and 39.1%. Total time of the crystallizations were 192 hours (8 days).

TABLE 9
Crystallization of iosimenol in glycols:

Experiment:

| | JM-230117A | JM-230117B |
|---|---|---|
| Starting Iosimenol, | 40 g | 40 g |
| Purity: | 97.76% | 97.76% |
| Diethylenglycol: | 70 mL | 0 mL |
| Triethylenglycol: | 0 mL | 70 mL |
| Water: | 5 mL | 5 mL |
| Tromethamol: | 0.25 g | 0.25 g |
| Tested temperature range: | 85-100° C. | 85-100° C. |
| Seeding: | 0.25 g | 0.25 g |
| Isopropanol added gradual during | 75 mL | 75 mL |
| Crystallization: | | |
| Crystallization observed: | yes | Yes |
| Total time of crystallization: | 192 hours | 192 hours |
| Purity of obtained crystal: | 94.80% | 98.47% |
| (% Area of HPLC): | | |
| Yield: | 48.0% | 39.1% |

### Example 22. Purification of Iosimenol (3)

Deionized iosimenol (80 grams, purity 97.67% HPLC area) was suspended in solvent mixture containing alkoxyalcohols (1-methoxy-2-propanol, 134 mL) and water (30 mL) and tromethamol (0.25 grams) in a 250 mL three-necked flask equipped with a condenser. The suspension was stirred and heated up to 90° C. until clear solution is obtained, then heated to reflux and seeds of iosimenol crystals (0.25 grams) were added. The mixture was maintained under reflux. The first crystals appeared after 16 hours. The course of crystallization was checked by monitoring of remaining dissolved iosimenol in sample of liquid phase and when remaining iosimenol stayed unchanged in two consecutive testing, the suspension was filtered at 80-90° C. The obtained solid material was washed with ethanol (100 mL) at 60-70° C. The HPLC purity of the iosimenol crystals (batch: JM-310117C) was 98.93% and the crystallization yields were 46.9%. Total time of the crystallizations was 144 hours (6 days). In a similar manner to the above procedure, another working example was prepared, the results of which are shown in Table 10.

TABLE 10
Crystallization of iosimenol in alkoxyalcohols:

Experiment:

| | JM-310117B | JM-310117C |
|---|---|---|
| Starting Iosimenol, | 40 g | 80 g |
| Purity: | 97.76% | 97.76% |
| 2-Ethoxyethano | 160 mL | 0 mL |
| 1-Methoxy-2-propanol | 0 mL | 134 mL |
| Water: | 30 mL | 30 mL |
| Tromethamol: | 0.25 g | 0.25 g |
| Tested temperature range: | 92-96° C. | 92-96° C. |
| Seeding: | 0.25 g | 0.25 g |
| Crystallization observed: | yes | yes |
| Total time of crystallization: | 144 | 144 |
| Purity of obtained crystal | 97.17% | 98.93% |
| (% Area of HPLC): | | |
| Yield: | 31.2% | 46.94% |

### Example 23. Purification of Iosimenol (4) Deionized

(5,5′-[(1,3-dioxo-1,3-propanediyl)bis[(2,3-dihydroxypropyl)imino]]bis[N-(2,3-dihydroxypropyl)-2,4,6-triiodo-1,3-benzenedicarboxamide]), iosimenol (10 grams, purity 97.67% HPLC area) was suspended in solvent mixture containing methanol (40 mL) and water (3 mL) and trometamol (0.1 grams) in a 300 mL Buchi glass pressure autoclaves equipped with a HPLC pump for gradient adding of methanol. The suspension was stirred and heated up to 90° C. until clear solution is obtained, then heated to 102-110° C. The mixture was maintained under elevated pressure (2.75-3.75 bars). The antisolvent (methanol, 75 mL) was added by HPLC pump (flow was 0.01 mL/min) in during 90 hours. The first crystals appeared after 6-8 hours. The course of crystallization was checked visually. After 10 hours of mixing crystallization mixture at 102-110° C., the suspension was cooled to temperature 65° C. During cooling of suspension was observed dissolution of part of crystals (about 30-40% of volume of crystal). The autoclave was opened and the suspension was filtered at 60-65° C. The obtained solid material was washed with ethanol (30 mL) at 60-70° C. The HPLC purity of the Iosimenol crystals (batch: JM-220917) was 99.19% and the crystallization yield was 45%. Total time of the crystallization was 111 hours.

TABLE 11
Crystallization of iosomenol in pressure autoclaves

| | Experiment: | | |
|---|---|---|---|
| | JM-220917 | JM-051017 | JM-191017 |
| Starting Iosimenol API, | 10 g, | 10 g, | 10 g, |
| purity: | 97.76% | 97.85% | 89.77% |
| Batch of starting | AK-160412 | AK-230212 | 00407004 |
| material: | | | |
| Methanol: | 40.0 mL | 58.0 mL | 160.0 mL |
| Water: | 3.00 mL | 0.44 mL | 0.30 mL |
| Trometamol: | 0.10 g | 0.10 g | 0.10 g |
| Gradient addition of | 75.0 mL | 67.0 mL | 0.0 mL |
| methanol | | | |
| Tested temperature range: | 102-110° C. | 95-102° C. | 98-105° C. |
| Tested overpressure | 2.75-3.75 | 2.0-2.75 | 2.5-3.35 |
| range: | Bar | Bar | Bar |
| Crystallization observed: | yes | yes | yes |
| Total time of | 111 hours | 138 hours | 93 hours |
| crystallization: | | | |
| Purity of obtained | 99.19% | 99.00% | 95.11% |
| crystal (% Area of HPLC): | | | |
| Yield: | 44.0% | 45.0% | 45.5% |

### Example 24. Purification of Iosimenol (5)

The first 250 mL three neck round-bottomed flask equipped with a mechanic stirrer was charged with iosimenol (lot number: 00407004, 14.78 grams, 0.010 mol) and 2-methoxyethanol (80 mL). The stirred mixture was heated to temperature of 80-85° C. The second 250 mL three neck round-bottomed flask equipped with a mechanic stirrer was charged with the anhydrous calcium chloride (1.28 grams, 0.011 mol) and 2-methoxyethanol (80 mL). The stirred mixture was heated to temperature of 80-85° C. and upper prepared solution of iosimenol in 2-methoxyethanol was continuously added to the reaction mixture during 5 minutes. The reaction mixture was heated for half hour at 90-95° C. The mixture was filtered at temperature of 80-90° C. The solids were collected on a filter funnel, followed by a wash using hot (80-90° C.) 2-methoxyethanol (2×20 mL). The wet cake was transferred to glass drying trays and the solid (13.45 grams) was dried in a rotary vacuum evaporator (P=15 mbar, T=70° C.) to afford 12.1 g (yield is 76.1%) of iosimenol calcium complex (batch number: JM-04001116/FKR). In a similar manner to the above procedure, some working examples were prepared, the results of which are shown in Table 12.

TABLE 12

| | Starting | | | | | | |
|---|---|---|---|---|---|---|---|
| | material - | | Condition of | | Prepared Iosimenol | | |
| | Iosimenol | | preparetion: | | calcium complex: | | |
| | Molar ratio | Starting | Temperature | | Iosimenol | | Molar ratio in |
| | Iosimenol:used | iosimenol | of | | complex purity | | separated complex |
| Experiment: | CaCl₂ | % HPLC area | reaction: | Filtration: | % HPLC area | Yield: | Iosimenol:CaCl₂ |
| JM- | 1:10 | 97.76% | 80-85° C. | 40-45° C. | 98.11% | 7.9% | 1:0.70 |
| 041116B | | | | | | | |
| JM- | 1:5 | 97.76% | 80-85° C. | 80-85° C. | 98.05% | 72.8% | 1:0.89 |
| 041116C | | | | | | | |
| JM- | 1:10 | 97.76% | 80-85° C. | 80-85° C. | 98.01% | 48.6% | 1:1.50 |
| 041116D | | | | | | | |
| JM- | 1:1 | 97.76% | 80-85° C. | 80-85° C. | 98.02% | 74.5% | 1:0.72 |
| 041116E | | | | | | | |
| JM- | 1:1 | 89.77% | 80-85° C. | 80-85° C. | 92.58% | 76.1% | 1:0.75 |
| 041116F | | | | | | | |

### INDUSTRIAL APPLICABILITY

The present invention provides effective processes of preparing iosimenol and each intermediate thereof of the present invention in a high yield. And, the present invention also provides effective purifications of iosimenol and each intermediate thereof in a high purity.

1. A process of preparing iosimenol shown in the following scheme:

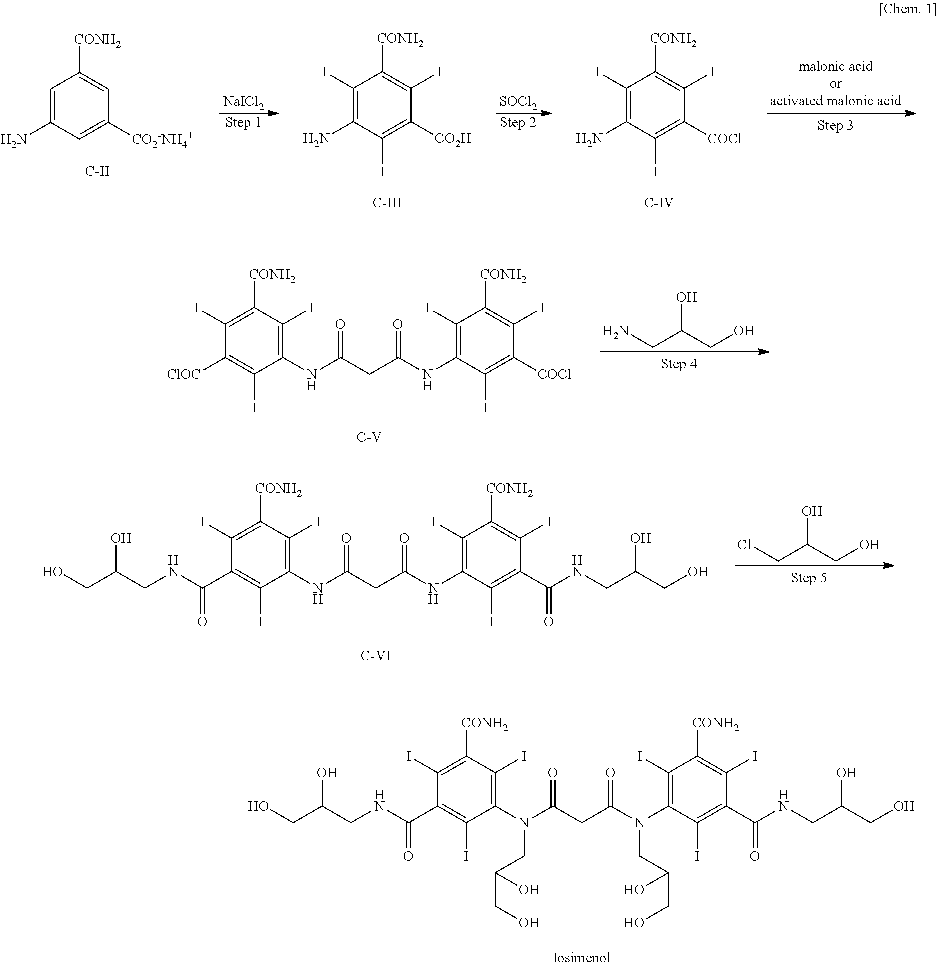

2. The process of claim 1, wherein the crude product C-III in Step 1 is purified by crystallization in a solvent comprising methanol and/or a mixture of methanol and water (methanol: 1-99 wt %) at 20 to 100° C.

3. The process of claim 1, wherein C-III in Step 2 is chlorinated with thionyl chloride in a solvent comprising ethyl acetate and/or toluene at reflux in the presence or without of catalytic amount of N,N-dimethylformamide.

4. The process of claim 1, wherein crude C-IV in Step 2 is purified using an anion exchange resin to remove organic impurities, where the resin is a polystyrene-based resin, a polyacrylate-based resin, preferably a benzene ethylene-divinylbenzene copolymer-based resin (a styrene-divinylbenzene copolymer-based resin).

5. The process of claim 1, wherein C-IV in Step 3 is coupled with malonic acid in the presence of phosphorus trichloride.

6. The process of claim 1, wherein C-IV in Step 3 is coupled with an activated malonic acid.

7. The process of claim 6, wherein as the activated malonic acid can be used its reactive ester or mixed anhydride which is in-situ prepared preferably by the addition of dicyclohexylcarbodiimide/N-hydroxybenztriazole and/or dicyclohexylcarbodiimide/hydroxysuccinimide and/or 1-ethyl-3-(3-dimethylaminopropyl)carbodiimide.HCl/N-hydroxybenztriazole and/or 1-propanephosphonic acid cyclic anhydride.

8. The process of claim 5, wherein the reaction is done in freshly distilled tetrahydrofuran or methyltetrahydrofuran.

9. The process of claim 5, wherein the crude product C-V is purified by stirring in a solvent comprising tetrahydrofuran, methyltetrahydrofuran, diethyl ether, dioxane, or a mixture thereof.

10. The process of claim 5, wherein the C-V is not dried and is used directly in the next step (Step 4).

11. The process of claim 1, wherein C-V in Step 4 reacts with 3-amino-propane-1,2-diol in an organic solvent in the presence of a base at 2-25° C.

12. The process of claim 11, wherein the organic solvent is N,N-dimethylformamide, in the presence of triethylamine.

13. The process of claim 11, wherein the C-VI reaction mixture is stirred with an anion exchange resin suspended in aqueous methanol to separate organic impurities.

14. The process of claim 13, wherein the anion exchange resin is a polystyrene-based resin, a polyacrylate-based resin, preferably a benzene ethylene-divinylbenzene copolymer-based resin (a styrene-divinylbenzene copolymer based resin).

15. The process of claim 11, wherein C—VI is separated and purified directly by precipitation from the reaction mixture by the addition of an organic solvent selected from methanol, ethanol, n-propanol, 2-propanol, or a combination thereof under pH 5-7.

16. The process of claim 11, wherein the precipitated C-VI is crystallized from a solvent mixture consisting water, acetone and acetic acid.

17. The process of claim 1, wherein C-VI reacts in Step 5 with an alkylating agent introducing 2,3-dihydroxypropyl group in the presence of an inorganic base in an organic solvent selected from N,N-dimethylformamide, N,N-dimethylacetamide, dimethyl sulfoxide, N-methyl-2-pyrrolidone, ethylene glycol, propylene glycol, glycerine, methanol, or a combination thereof in the presence of 2-methoxyethanol (0-99%).

18. The process of claim 17, wherein the alkylating agent introducing 2,3-dihydroxypropyl group is selected from the group consisting of 3-halo-propane-1,2-diol and glycidol.

19. The process of claim 17, wherein the alkylating agent introducing 2,3-dihydroxypropyl group is 3-halo-propane-1,2-diol.

20. The process of claim 17, wherein the alkylating agent introducing 2,3-dihydroxypropyl group is added into the stirred reaction mixture in one or more portion during reaction time.

21. The process of claim 17, wherein the reaction temperature is 10-60° C.

22. The process of claim 17, wherein the inorganic base is selected from the group consisting of an alkali metal hydroxide and an alkaline earth metal hydroxide.

23. The process of claim 17, wherein the inorganic base is lithium hydroxide, calcium hydroxide, sodium hydroxide, potassium hydroxide, or a mixture thereof.

24. The process of claim 17, wherein the reaction to prepare iosimenol is done in the presence of a metal halide besides an inorganic base.

25. The process of claim 24, wherein the metal halide is selected from the group consisting of CaCl₂, ZnCl₂, MgCl₂, CaBr₂, ZnBr₂ and MgBr₂.

26. A process of preparing C-III shown in the following scheme: 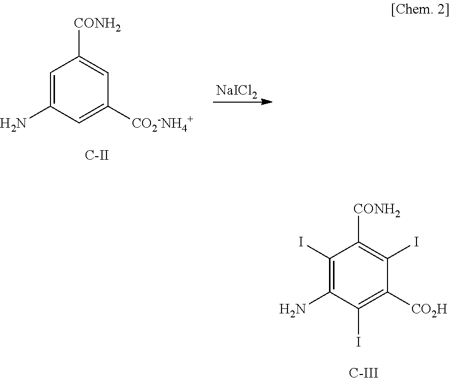
wherein the crude product C-III is purified by crystallization in a solvent comprising methanol or a mixture of methanol and water (methanol: 1-99 wt %) at 20 to 100° C.

27. A crystal of C-III methanol solvate of the following formula: 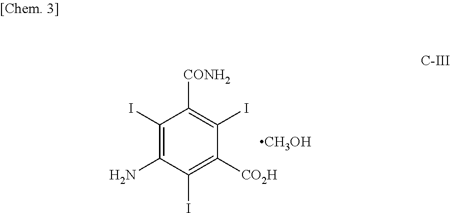
which is characterized by a powder x-ray diffraction pattern having four or more 2θ±0.2 peaks and selected from about 12.2°, 12.8°, 15.0°, 21.1°, 21.4°, 22.7°, 24.6°, 25.3°, 27.2°, 31.0°, 31.2°, 33.4°, and 33.9°, wherein measurement of said crystal is at a temperature of about 293 K.

28. A crystal of C-III methanol solvate of the following formula: 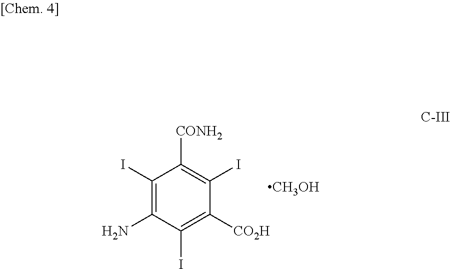
which is characterized by unit cell parameters at T=293K substantially equal to the following: a=17.000 (1) Å, b=13.896 (1) Å, c=12.597 (1) Å, unit-cell volume V=2975.9 Å³ and an orthorhombic space group Pbca.

29. A process of preparing C-IV shown in the following scheme: 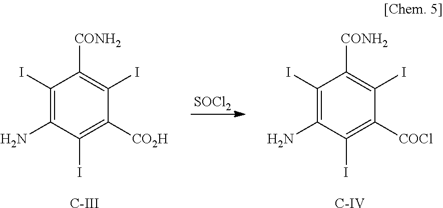
wherein C—III is chlorinated with thionyl chloride in a solvent comprising ethyl acetate and/or toluene at reflux in the presence or without of catalytic amount of N,N-dimethylformamide.

30. The process of claim 29, wherein the crude C-IV is purified using an anion exchange resin to remove organic impurities, where the resin is a polystyrene-based resin, a polyacrylate-based resin, preferably a benzene ethylene-divinylbenzene copolymer-based resin (a styrene-divinylbenzene copolymer based resin).

31. A process of preparing C-V shown in the following scheme: 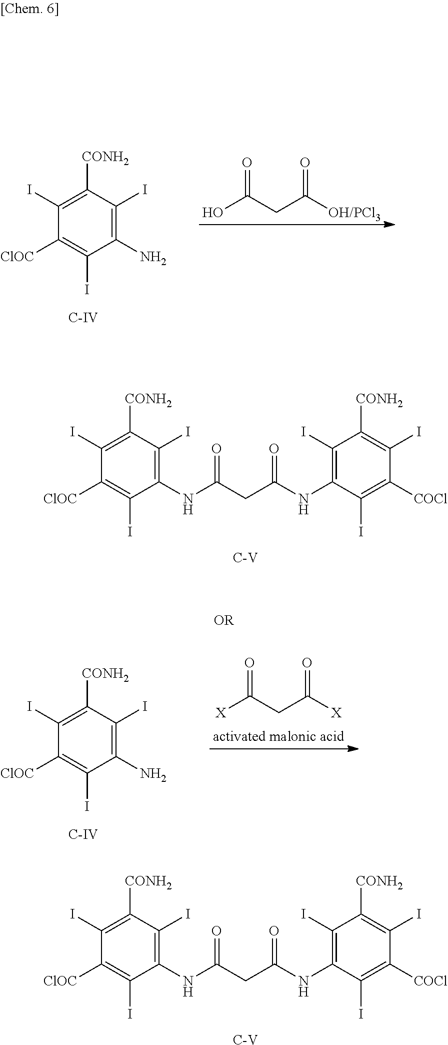
wherein C—IV is coupled with malonic acid in the presence of phosphorus trichloride, or with an activated malonic acid.

32. The process of claim 31, wherein C-IV in Step 3 is coupled with an activated malonic acid.

33. The process of claim 32, wherein as the activated malonic acid can be used its reactive ester or mixed anhydride which is in-situ prepared preferably by addition of dicyclohexylcarbodiimide/N-hydroxybenztriazole and/or dicyclohexylcarbodiimide/hydroxysuccinimide and/or 1-ethyl-3-(3-dimethylaminopropyl)carbodiimide.HCl/N-hydroxybenztriazole and/or 1-propanephosphonic acid cyclic anhydride.

34. The process of claim 31, wherein the reaction is done in freshly distilled tetrahydrofuran or methyltetrahydrofuran.

35. The process of claim 31, wherein the crude product C-V is purified by stirring in a solvent comprising tetrahydrofuran, methyltetrahydrofuran, diethyl ether, dioxane or a mixture thereof.

36. The process of claim 31, wherein the C-V is not dried and is used directly in the next reaction step.

37. A process of preparing C-VI shown in the following scheme: 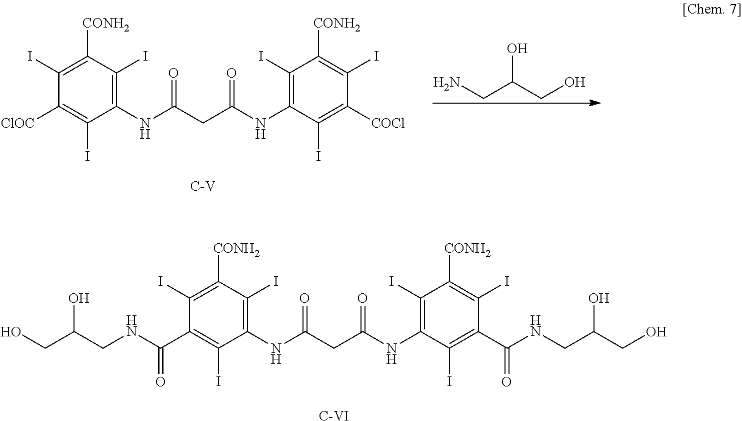
wherein C-V reacts with 3-amino-propane-1,2-diol in organic solvent in the presence of a base at 2-25° C.

38. The process of claim 37, wherein the organic solvent is N,N-dimethylformamide, in the presence of trimethylamine.

39. The process of claim 37, wherein C-VI reaction mixture is stirred with an anion exchange resin suspended in aqueous methanol to separate organic impurities.

40. The process of claim 39, wherein the anion exchange resin is a polystyrene-based resin, a polyacrylate-based resin, preferably a benzene ethylene-divinylbenzene copolymer-based resin (a styrene-divinylbenzene copolymer based resin).

41. The process of claim 37, wherein C-VI is separated and purified directly by precipitation from the reaction mixture by addition of an organic solvent selected from methanol, ethanol, n-propanol, 2-propanol or a combination thereof under pH 5-7.

42. The process of claim 37, wherein precipitated C-VI is crystallized from a solvent mixture consisting water, acetone and acetic acid.

43. A process of preparing iosimenol shown in the following scheme: 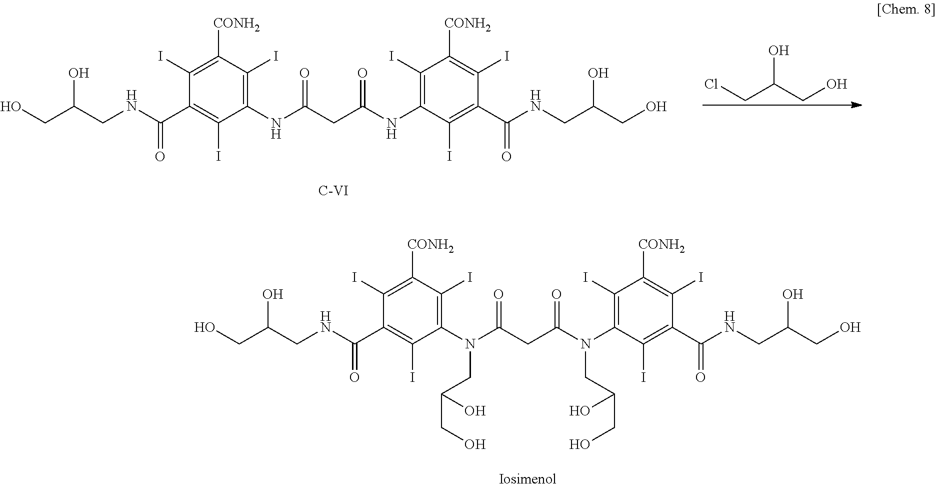 [SMILES: NC(=O)c1c(I)c(NC(=O)CC(=O)Nc2c(I)c(C(N)=O)c(I)c(C(=O)NCC(O)CO)c2I)c(I)c(C(=O)NCC(O)CO)c1I ; OCC(O)CCl ; "C-VI" ; NC(=O)c1c(I)c(C(=O)NCC(O)CO)c(I)c(N(CC(O)CO)C(=O)CC(=O)N(CC(O)CO)c2c(I)c(C(N)=O)c(I)c(C(=O)NCC(O)CO)c2I)c1I]
wherein C-VI reacts with an alkylating agent introducing 2,3-dihydroxypropyl group in the presence of an inorganic base in an organic solvent selected from N,N-dimethylformamide, N,N-dimethylacetamide, dimethyl sulfoxide, N-methyl-2-pyrrolidone, ethylene glycol, propylene glycol, glycerine, methanol or a combination thereof in the presence of 2-methoxyethanol (0-99%).

44. The process of claim 43, wherein the alkylating agent introducing 2,3-dihydroxypropyl group is selected from the group consisting of 3-halo-1,2-propane-1,2-diol and glycidol.

45. The process of claim 43, wherein the alkylating agent introducing 2,3-dihydroxypropyl group is 3-halo-propane-1,2-diol.

46. The process of claim 43, wherein the alkylating agent introducing 2,3-dihydroxypropyl group is added into the stirred reaction mixture in one or more portion during the reaction time.

47. The process of claim 43, wherein the reaction temperature is 10-60° C.

48. The process of claim 43, wherein the inorganic base is selected from the group consisting of an alkali metal hydroxide and an alkaline earth metal hydroxide.

49. The process of claim 43, wherein the inorganic base is lithium hydroxide, calcium hydroxide, sodium hydroxide, potassium hydroxide or a mixture thereof.

50. The process of claim 43, wherein the reaction to prepare iosimenol is done in the presence of a metal halide besides an inorganic base.

51. The process of claim 50, wherein the metal halide is selected from the group consisting of CaCl₂, ZnCl₂, MgCl₂, CaBr₂, ZnBr₂ and MgBr₂.

52. A process of preparing iosimenol shown in the following scheme:

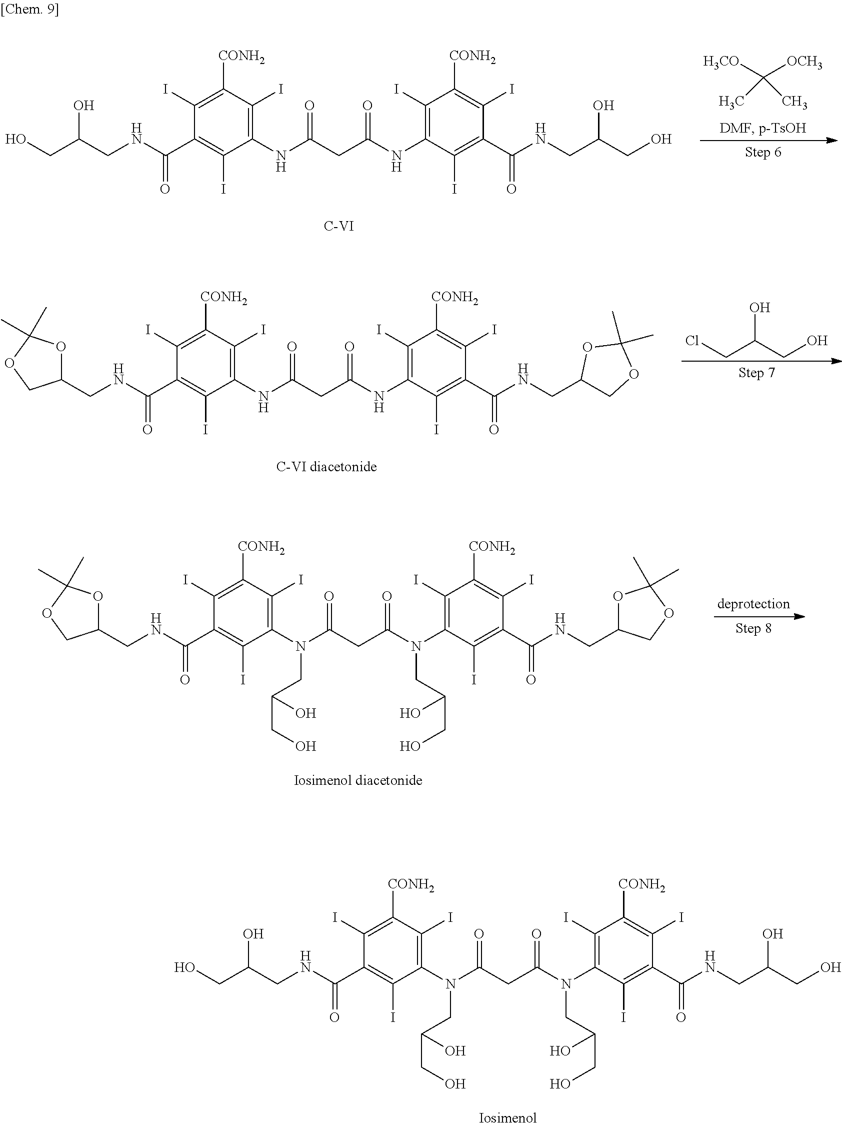

53. The process of claim 52, wherein C—VI is protected with 2,2-dimethoxypropane in N,N-dimethylformamide, in the presence of an acidic catalyst.

54. The process of claim 52, wherein C-VI diacetonide is crystallized from the reaction mixture by adding water.

55. The process of claim 52, wherein a crystal of C-VI diacetonide is obtained in high purity and high yield.

56. The process of claim 52, wherein C-VI diacetonide reacts with an alkylating agent introducing 2,3-dihydroxypropyl group in the presence of an inorganic base in an organic solvent selected from N,N-dimethylformamide, N,N-dimethylacetamide, dimethyl sulfoxide, N-methyl-2-pyrrolidone, ethylene glycol, propylene glycol, glycerine, methanol, or a combination thereof, and/or their mixtures with methoxyethanol (0-99%).

57. The process of claim 52, wherein the alkylating agent introducing 2,3-dihydroxypropyl group is selected from the group consisting of 3-halo-propane-1,2-diol and glycidol.

58. The process of claim 52, wherein the alkylating agent introducing 2,3-dihydroxypropyl group is 3-halo-propane-1,2-diol.

59. The process of claim 52, wherein the alkylating agent introducing 2,3-dihydroxypropyl group is added into the stirred reaction mixture in one or more portion during the reaction time.

60. The process of claim 52, wherein the reaction temperature is 10-60° C.

61. The process of claim 52, wherein the inorganic base is selected from the group consisting of an alkali metal hydroxide and an alkaline earth metal hydroxide.

62. The process of claim 52, wherein the inorganic base is lithium hydroxide, calcium hydroxide, sodium hydroxide, potassium hydroxide, or a mixture thereof.

63. The process of claim 52, wherein the reaction to prepare iosimenol diacetonide is done in the presence of a metal halide besides an inorganic base.

64. The process of claim 63, wherein the metal halide is selected from the group consisting of CaCl₂, ZnCl₂, MgCl₂, CaBr₂, ZnBr₂ and MgBr₂.

65. The process of claim 52, wherein iosimenol diacetonide is obtained by crystallization from an organic solvent selected from methanol, ethanol, n-propanol, 2-propanol, or a combination thereof.

66. The process of claim 52, wherein iosimenol is obtained by deprotection of iosimenol diacetonide in an aqueous or methanolic, or ethanolic solution, or a methanol-water or ethanol-water in the presence of a strong acid.

67. A process of purifying iosimenol, wherein a crude iosimenol is purified by crystallization in (i) binary or tertiary solvent-mixture selected from 2-methoxyethanol, 1-methoxy-2-propanol, and alcohols which a selected from methanol, ethanol, 2-propanol, n-butanol and/or 2-butanol (ii) diethylene glycol and/or triethylene glycol, or (iii) 2-ethoxyethanol and/or 1-methoxy-2-propanol, in the presence of water.

68. A process of purifying iosimenol which is done from a saturated or supersaturated solution of said compound comprising:
Step 1: suspending the deionized iosimenol in a solvent mixture comprising one or more organic solvent and water,
Step 2: subjecting the mixture to heat and/or ultrasonic to make the mixture completely dissolved,
Step 3: continuing to subject the solution to the same or different heat and/or ultrasonic to deposit a crystal,
Step 4: continuous addition of a solvent or a solvent mixture, or adding of a solvent or a solvent mixture in individual portions during the crystallization process,
Step 5: collecting the resulting crystal on a filter.

69. The process of claim 68, wherein the heating in Step 2 and/or Step 3 and/or Step 4 is done with microwave.

70. The process of claim 68, wherein the organic solvent in Step 1 and Step 4 comprises one or more C₁-C₆ linear or branched alkanols or alkoxyalkanols, C₂-C₈ aliphatic ethers, C₄-C₆ cyclic ethers, and/or glycols.

71. The process of claim 68, wherein the organic solvent in Step 1 and Step 4 is selected from the group consisting of methanol, ethanol, n-propanol, 2-propanol, n-butanol, i-butanol, sec-butanol, tert-butanol, pentanols including isoamylalcohols, hexanols, 2-methoxyethanol, 2-ethoxyethanol, 1-methoxy-2-propanol, 2-isopropoxyethanol, ethylene glycol, diethylene glycol and triethylene glycol.

72. The process of claim 68, wherein the solvent mixture in Step 1 and Step 4 contains up to 20% water.

73. The process of claim 68, wherein the crystallization process in Step 3 may be initiated by adding a seed of iosimenol crystal while or after the temperature is raised.

74. The process of claim 68, wherein trometamol is used to buffer pH during the crystallization process.

75. The process of claim 68, wherein Steps 2, 3 and 4 are done at 70° C.-140° C. and at the pressure of 0-10 bars.

76. The process of claim 68, wherein the concentration of iosimenol as the starting material in Step 1 and Step 4 is 10 w/v %-60 w/v %.
